(19) United States
(12) Patent Application Publication
(10) Pub. No.: US 2009/0099149 A1
(43) Pub. Date: Apr. 16, 2009

(54) BIOADHESIVE FILM

(75) Inventors: Xiuying LIU, Glen Rock, NJ (US); John KRESEVIC, New Windsor, NY (US)

Correspondence Address: Pepper Hamilton LLP/Wyeth, 400 Berwyn Park, 899 Cassatt Road, Berwyn, PA, 19312-1183, US

(73) Assignee: Wyeth, Madison, NJ (US)

(21) Appl. No.: 12/245,000

(22) Filed: Oct. 3, 2008

Related U.S. Application Data
(60) Provisional application No. 60/979,180, filed on Oct. 11, 2007.

(51) Int. Cl. A61K 31/565; A61K 47/10; A61K 47/32; A61K 47/38

(52) U.S. Cl. 514/182; 514/772; 514/772.6; 514/781

(57) ABSTRACT

Bioadhesive films for delivery of active agent to the mucosa are disclosed. Particularly, bioadhesive films for treating the vaginal mucosa are disclosed.

### CROSS REFERENCE TO RELATED APPLICATIONS

This application claims priority benefit of U.S. Provisional Application Ser. No. 60/979,180 filed Oct. 11, 2007, which is incorporated by reference herein in its entirety.

### FIELD OF INVENTION

The invention relates to a bioadhesive film for delivering an active pharmaceutical agent. More particularly, some embodiments of the invention relate to bioadhesive films for delivering an active agent via contact with the mucosa.

### BACKGROUND OF THE INVENTION

Mucosa is moist tissue that lines some organs and body cavities throughout the body, including, for example, the nose, mouth, lungs, digestive tract, urethra, and vagina. Particularly, mucosa lines body passages that have contact with outside air. Glands along the mucosa release mucus, making it difficult for biological or synthetic materials to hold or adhere to the mucosa. Thus, one key to delivering active agents at the mucosa is bioadhesion.

Other delivery forms have been used to administer active agents to or via the mucosa, but have some disadvantages over a bioadhesive film administration, which provide localized effects. Oral, topical and transdermal dosage forms are systemically absorbed. Therefore, they could have unwanted effects on other body parts. Creams often need a special applicator to use, are messy to administer, and tend to run. In the context of treating the vaginal mucosa, vaginal rings introduce a non-degradable foreign body in the organism and require insertion and removal manipulations. Vaginal tablets require special applicator to administer. Thus, there is a need for bioadhesive films that can be applied discreetly and neatly, with minimal running to ensure long term retention.

### SUMMARY OF THE INVENTION

According to some embodiments, the invention provides bioadhesive films for administration of an active agent via the body's mucosa, particularly the vaginal mucosa.

According to some embodiments, the invention provides a bioadhesive film comprising a film forming polymer, a plasticizer, and a bioadhesive agent.

According to some embodiments, the invention provides a bioadhesive film comprising a film forming polymer, a plasticizer, a bioadhesive agent, and a pharmaceutically active agent.

According to some embodiments, the invention provides a pharmaceutically acceptable bioadhesive film comprising:

About 65-95% film forming polymer by weight;

About 5-20% plasticizer by weight; and

Less than about 10% bioadhesive agent by weight;

and a pharmaceutically active agent.

In some embodiments, the film forming polymer is selected from polyvinyl alcohol (PVA), polyethylene oxide, hydroxypropyl cellulose (HPC), hydroxyethyl cellulose (HEC), methylcellulose (MC), and hydroxypropyl methylcellulose (HPMC), copovidone, gelatin, maltodextrin, xanthan gum, guar gum, polymethacrylate, povidone, and mixtures thereof.

In some embodiments, the plasticizer is selected from glycerin, polyethylene glycols, propylene glycol, mannitol, sorbitol, dibutyl phthalate, tributyl citrate, dimethyl phthalate, pyrrolidones, and mixtures thereof.

In some embodiments, the bioadhesive agent is selected from polyacrylic acid derivatives, cellulose derivatives, substances of nature origin, protein, and mucilaginous substances from edible vegetables, and mixtures thereof.

In some embodiments, the bioadhesive agent is a polyacrylic acid derivative selected from high-molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, and combinations thereof.

In some embodiments, the bioadhesive agent is Carbomer Homopolymer Type B USP/NF(CARBOPOL 974P), and polycarbophil USP/NF (NOVEON AA-1). In some embodiments, the bioadhesive agent is a cellulose derivative, e.g. alkylcelluloses, hydroxyalkylcelluloses, cellulose ethers, cellulose esters, nitrocelluloses, etc.

In some embodiments, the bioadhesive is a substance of nature origin (e.g. chitosans, guar gum, xanthan gum, carrageenan, pectin, sodium alginate, dextrans, lectins, aminated gelatin, aminated pectin, hyaluronic acid, inulin, etc . . . ) In some embodiments, the active agent is selected from anti-infective agents, spermicides, estrogens, progestins; deodorizers, and combinations thereof. In some embodiemtns, the active agent is an anti-infective agents selected from antibiotics, sulfonamides, antivirals, antifungals, antiprotozoans, and mixtures thereof.

In some embodiments, the active agent is a spermicide selected from nonoxynol-9, octoxynol-9, benzalkonium chloride, ricinoleic acid, and phenol mercuric acetates, and combinations thereof.

In some embodiments, the active agent is one or more estrogen selected from conjugated estrogens, synthetic conjugated estrogens, esterified estrogens, 17b-estradiol, estradiol acetate, estropipate, and estradiol hemihidrate.

In some embodiments, the active agent is a progestin selected from medroxyprogesterone acetate, norethindrone, norgestel, megestrol acetate, progesterone, levonorgestrel, drospirenone, norgestimate, and methyltestosterone, and combinations thereof.

Some embodiments of the invention provide a bioadhesive film comprising:

65-90% polyvinyl alcohol by weight;

5-20% glycerin by weight;

5-8% Carbomer Homopolymer Type B USP/NF by weight; and

0.01 -0.50% conjugated estrogen concentrate (16mg/g) by weight.

Some embodiments of the invention provide a bioadhesive film further comprising an amount of pH buffer sufficient to adjust the film pH to about 7.4.

Some embodiments of the invention provide a bioadhesive film comprising:

About 65-95%, by weight film forming polymer selected frompolyvinyl alcohol (PVA), polyethylene oxide, hydroxypropyl cellulose (HPC), hydroxyethyl cellulose (HEC), methylcellulose (MC), and hydroxypropyl methylcellulose (HPMC), copovidone, gelatin, maltodextrin, xanthan gum, guar gum, polymethacrylate, povidone, and mixtures thereof;

About 5-20%, by weight, plasticizer selected from glycerin, polyethylene glycols, propylene glycol, mannitol, sorbitol, dibutyl phthalate, tributyl citrate, dimethyl phthalate, pyrrolidones, and mixtures thereof and

Less than about 10%, by weight, bioadhesive agent selected from high-molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, and combinations thereof; and a pharmaceutically active agent selected from anti-infective agents, spermicides, estrogens, progestins; deodorizers, and combinations thereof.

### BRIEF DESCRIPTION OF THE DRAWINGS

FIG. 1 is a graph depicting the Influence of Levels of PVA, Glycerin, and Carbopol 974P on Tensile Strength of Films in accordance with an embodiment of the invention Based on Quadratic Model

FIG. 2 is a graph depicting the Influence of Levels of PVA, Glycerin, and Carbopol 974P on Percent Elongation of Films in accordance with an embodiment of the invention Based on Linear Model

FIG. 3 is a graph depicting the Influence of Levels of PVA, Glycerin, and Carbopol 974P on Elastic Modulus of Films in accordance with an embodiment of the invention Based on Quadratic Model

FIG. 4 is a graph depicting the Influence of Levels of PVA, Glycerin, and Noveon AA-1 on Tensile strength of Films in accordance with an embodiment of the invention Based on Linear Model

FIG. 5 is a graph depicting the Influence of Levels of PVA, Glycerin, and Noveon AA-1 on Percent Elongation of Films in accordance with an embodiment of the invention Based on Linear Model

FIG. 6 is a graph depicting the Influence of Levels of PVA, Glycerin, and Noveon AA-1 on Elastic Modulus of Films in accordance with an embodiment of the invention Based on Linear Model

FIG. 7 is a graph depicting the Influence of Levels of PVA, Glycerin, and Carbopol 974P on Visual Dissolution Time of Films in accordance with an embodiment of the invention Based on Special Cubic Model

FIG. 8 is a graph depicting Influence of Levels of PVA, Glycerin, and Noveon AA-1 on Visual Dissolution Time of Films in accordance with an embodiment of the invention Based on Quadratic Model

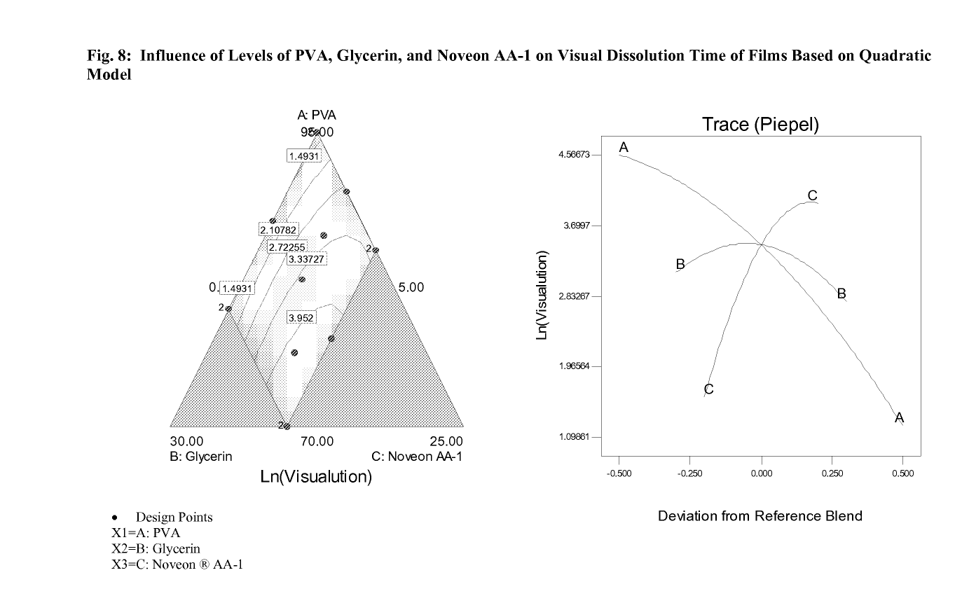

FIG. 9 is a graph depicting the Influence of Levels of PVA, Glycerin, and Carbopol 974P on Bioadhesive Properties (Holding Time) of Films in accordance with an embodiment of the invention Based on Quadratic Model

FIG. 10 is a graph depicting the Influence of Levels of PVA, Glycerin, and Noveon AA-1 on Bioadhesive Properties (Holding Time) of Films in accordance with an embodiment of the invention Based on Special Cubic Model

FIG. 11 is a graph depicting Dissolution Profiles of CE Films in accordance with an embodiment of the invention

FIG. 12 is a graph depicting the Influence of pH on CE Dissolution Profiles from CE Films in accordance with an embodiment of the invention

### DETAILED DESCRIPTION OF THE INVENTION

Bioadhesive films for administering an active agent at the mucosa are disclosed herein. The film comprises a film-forming agent, a plasticizer, an adhesive enhancer, and an adhesive.

Bioadhesion (or mucoadhesion) is generally defined as the ability of a biological or synthetic material to adhere to a mucous membrane, resulting in adhesion of the material to the tissue for a protracted period of time. Bioadhesion may enhance drug bioavailability due to the longer period of time in which the bioadhesive dosage form is in contact with the absorbing tissue versus a standard dosage form, such as tablet, sphere, capsule or film. To adhere to a mucous membrane, interaction, intermixing and/or amalgamation between a material and mucus, which is a highly hydrated, viscous anionic hydrogel layer protecting the mucosa, is needed.

Given the location of the various mucosa, special considerations should be taken into account when developing a bioadhesive film. Properties such as rigidity, elasticity, tensile strength, solubility, etc. become important in film design and can be controlled and adjusted by manipulating the various concentrations or type of components used.

The bioadhesive film of the invention comprises a film-forming agent, a plasticizer, and an adhesive. A variety of pharmaceutically active agents may be incorporated into the film.

A suitable film-forming agent includes, but is not limited to polymers, such as polyvinyl alcohol (PVA), polyethylene oxide, hydroxypropyl cellulose (HPC), hydroxyethyl cellulose (HEC), methylcellulose (MC), and hydroxypropyl methylcellulose (HPMC), copovidone, gelatin, maltodextrin, xanthan gum, guar gum, polymethacrylate, and povidone can also be employed. PVA was studied in this research as a film-forming agent.

Film forming agent is present in the film at about 60-95% by weight. In some embodiments, the film forming agent is about 65-80% by weight. In some embodiments, the film forming agent is present at about 65% or 75% by weight.

Polyvinyl alcohol (PVA) is a water-soluble synthetic resin that is prepared by polymerization of vinyl acetate, followed by partial hydrolysis of the resulting ester in the presence of an alkaline catalyst. The number of acetate groups in Polyvinyl Alcohol is determined by the degree of hydrolysis. The physical characteristics of PVA vary depending on the degree of polymerization and hydrolysis. PVA is classified into grades of partially and fully hydrolyzed polymers. PVA is used primarily in topical pharmaceutical and ophthalmic formulations. It is used as stabilizing agent for emulsions. PVA is also an excellent film-forming agent. It is widely used in the pharmaceutical film coating.

Suitable plasticizers are known in the art, including but not limited to glycerin, polyethylene glycols, propylene glycol, mannitol, sorbitol, dibutyl phthalate, tributyl citrate, dimethyl phthalate, pyrrolidones, and combinations thereof. Glycerin is commonly used as a plasticizer for soft-shell capsules and film formulations, and is appropriate for use in the bioadhesive films described herein.

Plasticizer is present in the film from about 2-20% by weight. In some embodiments, plasticizer is present from about 5-20% by weight. In some embodiments, the plasticizer is present from about 10-20% by weight.

The examples herein are directed to the use of glycerin, but as with the other components, the relative amounts and type of plasticizer used, will vary depending upon the desired properties of the film and the effects of a particular plasticizer.

Suitable adhesives include, but are not limited to, polyacrylic acid derivatives, cellulose derivatives, substances of natural origin, protein, and mucilaginous substances from edible vegetables. Combinations of these adhesives can also be used for increasing the mucoadhesive properties of the film.

The bioadhesive is present in the film at less than about 20%. In some embodiments, the bioadhesive is present at less than 10% by weight. In some embodiments, the bioadhesive is present at about 7.5% by weight.

Polyacrylic acid derivatives include, but are not limited to, high molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, or combinations thereof.

The examples herein focus on specific high molecular weight cross-linked acrylic acid polymers. However, it will be recognized that other adhesives can be used, with their relative concentrations being adjusted to account for the desired film properties and the properties imparted by a particular agent. In particular, the bioadhesive properties of CARBOPOL 974P and NOVEON AA-1 in the film were investigated when combined with PVA and glycerin.

CARBOPOL polymers and NOVEON polycarbophils are very high molecular weight polymers of acrylic acid, crosslinked with polyalkenyl ethers or divinyl glycol 3. When exposed to a pH environment above 4-6, the polymers swell up to 1000 times their original volume (and ten times their original diameter) in water to form a gel, providing a large adhesive surface area for maximum contact with the mucin. Above their pKa of 6±0.5, the carboxylate groups on the polymer backbone ionize, resulting in repulsion between the anions and further increasing the swelling of the polymer. Crosslinked polymers do not dissolve in water, but form colloidal gel dispersions.

CARBOPOL 974P was introduced specifically for use in oral and mucoadhesive contact applications such as controlled release tablets, oral suspensions and bioadhesives. In addition, CARBOPOL 974P provides the thickening, suspending, and emulsification properties to high viscosity systems for topical applications. CARBOPOL 974P meets the United States Pharmacopeia/National Formulary (USP/NF) monograph for Carbomer Homopolymer Type B, the European Pharmacopeia (Ph. Eur.) monograph for Carbomers and the Japanese Pharmaceutical Excipients (JPE) monograph for Carboxyvinyl Polymer. (Note: The previous USP/NF compendial name for this product was Carbomer 934P.)

NOVEON AA-1 polycarbophil, USP is a high molecular weight acrylic acid polymer crosslinked with divinyl glycol. It provides excellent bioadhesive properties and has been used extensively to enhance the delivery of active ingredients to various mucous membranes. NOVEON AA-1 polycarbophil USP meets the United States Pharmacopeia/National Formulary (USP/NF) monograph for Polycarbophil.

Cellulose derivatives include but are not limited to alkylcelluloses, hydroxyalkylcelluloses, cellulose ethers, cellulose esters, nitrocelluloses, etc.

Substances of natural origin including chitosans, guar gum, xanthan gum, carrageenan, pectin, sodium alginate, dextrans, lectins, aminated gelatin, aminated pectin, hyaluronic acid, inulin, etc. may also be used as bioadhesives.

Proteins such as zein, serum albumin, and collagen can also be used as suitable bioadhesives.

The relative amount of the bioadhesive will vary according to desired film properties and the properties imparted by a particular agent. For example, the type and amount of bioadhesive can affect both adhesion strength and release rate of active agent (in addition to other properties). The relative amount of bioadhesive agent will be selected in accordance with the effect of a particular bioadhesive on the desired film properties.

A variety of active agents can be incorporated in the bioadhesive film. Suitable active agents which can be delivered with the film include, but are not limited to:

Analgesics and/or anesthetics such as aspirin, non-steroidal anti-inflammatory drugs (NSAIDS), COX-2 inhibitors, Opiates and morphinomimetics, lidocaine, prilocaine, or other analgesics and anesthetics known in the pharmaceutical arts, as well as combinations thereof.

Anti-infective agents such as antibiotics, sulfonamides, antivirals, antifungals, and antiprotozoan;

Spermicides such as nonoxynol-9, octoxynol-9, benzalkonium chloride, ricinoleic acid, and phenol mercuric acetates;

Estrogens, such as conjugated estrogens, synthetic conjugated astrogens, esterified estrogens, 17β-estradiol, estradiol acetate, estropipate, and estradiol hemihyd rate;

Progestin, such as medroxyprogesterone acetate, norethindrone, norgestel, megestrol acetate, progesterone, levonorgestrel, drospirenone, norgestimate, and methyltestosterone; and

deodorizers.

The amount of active agent used will depend upon its pharmaceutically effective dose and film properties. Film properties can be adjusted by altering levels of film forming agent, plasticizer, and bioadhesive agent to achieve the desired film properties.

The use of conjugated estrogens (CE) is exemplified herein. Particularly, a CE concentrate containing 16 mg CE/g was used. Other formulations may also be incorporated, making suitable adjustments in relative amounts depending on the desired film properties.

Although the examples focus on delivery of CE, the amount and type of active ingredients is not limited to CE or estrogens, and may encompass additional active agents or combination of agents, without leaving the scope and spirit of the invention. CE are used for estrogen replacement therapy (ERT), which is beneficial for symptomatic relief of hot flushes, genital atrophy, and for the prevention of postmenopausal osteoporosis. In some embodiments, CE concentration is about 0.15% by weight, when using a CE concentrate having about 16 mg CE/g. CE concentration can range from about 0.01-0.50% by weight. In some embodiments, CE concentration is about 12-17% by weight. (do you mean 0.12-0.17%?)

Bioadhesive films of the invention are well-suited for vaginal applications because vaginal films will provide localized effects. Oral, topical and transdermal dosage forms will be systemically absorbed. Therefore, they can have unwanted effects on other body parts. Vaginal creams often require a special applicator and are messy to administer. Then, after application, they tend to run. Vaginal rings will bring a non-degradable foreign body in the organism. They require insertion and removal manipulations. Vaginal tablets require special applicator to administer.

On the other hand, vaginal films of this invention can be applied discreetly. They can be administered without creating a messy condition. Since they are bioadhesive in nature, they can allow the film to adhere to the mucous membrane with a slight flow. Thus ensuring long term retention.

The desired film properties are determined by the type of application and achieved by the kind and quantity of each component used. Accordingly, the relative amounts of each agent can be adjusted as needed to achieve the desired film properties.

As will be seen below in the examples drawn to placebo formulations, desired film characteristics can be achieved generally with bioadhesive films comprising:

70-95% film forming agent by weight;

5-20% plasticizer by weight; and

Less than 10% bioadhesive by weight.

Effective amounts of active agent can be added to this general formulation. Addition of active agent itself will also affect the film characteristics, so the amount of each component can be adjusted accordingly to achieve the desired results.

It should be kept in mind that lowering the content of plasticizer increase the brittleness of the film, while lower bioadhesive will lower the bioadhesive effect. Films containing plasticizer as low as 2% can be made, with an appropriate adjustment in bioadhesive, other components, or moisture level to adjust the brittleness to a desired level. Likewise, bioadhesive in amounts as high as 20% can be used, but may need to be diluted because they tend to be highly viscous. The exact ranges of each component can vary since the addition of one often counterbalances the other and desirable film properties can be achieved in various combinations. The combinations set forth herein are exemplary

Particularly, formulations comprising 70-95% PVA, 5-20% glycerin and less than 10% high molecular weight cross-linked acrylic acid polymers (CARBOPOL and NOVEON) were prepared and tested, without active agent, for various properties discussed below.

With this basic construct in mind, films were developed adding an active agent. Generally, bioadhesive films comprising 65-95% film forming polymer by weight; 5-20% plasticizer by weight; <10% a bioadhesive agent by weight; and a pharmaceutically active agent provide a suitable range of properties.

The examples below use a CE concentrate as active agent. Generally, such a bioadhesive film comprises:

65-90% polyvinyl alcohol by weight;

5-20% glycerin by weight;

5-8% CARBOPOL 974P by weight; and

0.10-0.20% conjugated estrogen concentrate (16 mg/g) by weight.

In some embodiments, the pH can be adjusted with the addition of a pH buffer, such as tris(hydroxymethyl)-aminomethane or triethanolamine. In some embodiments, the pH is adjusted to about 7.4. In some embodiments, the pH is adjusted through the addition of about 10% by weight (hydroxymethyl)-aminomethane or triethanolamine, or other suitable pH buffer.

Additional or different active agents will have their own affect on the film properties. However, as discussed below, the properties can be manipulated by increasing or decreasing the relative amount of one or more of the remaining components. Such manipulations are considered part of the invention herein and can readily be ascertained from the teachings herein.

### EXAMPLES

Studies on various formulations encompassing several levels related to glycerin and the high molecular weight cross-linked acrylic acid polymers, Carbomer Homopolymer Type B USP/NF(CARBOPOL 974P), and polycarbophil USP/NF (NOVEON AA-1) were studied/evaluated. Studies incorporating an experimental design package (Design Expert® 6.09 software) were carried out in order to evaluate the influences of levels of PVA, glycerin, and CARBOPOL 974P/NOVEON AA-1 on the film properties.

### Materials and Methods

### Experimental Design for Placebo Film Formulations

In order to study the influence of ingredients on the film properties, a D-optimal mixture experimental design was used to prepare systematic model formulations, which were composed of three formulation variables: level of PVA (X1), level of Glycerin (X2), and level of either CARBOPOL 974P or NOVEON AA-1 (X3). For this study, PVA 87-89% partially hydrolyzed with molecular weight of 11000-31000 was used. The causal factors are listed in Table 1 and Table 2 for formulations containing CARBOPOL and NOVEON, respectively. According to the D-optimal mixture model, 14 model formulations, which include 6 estimate formulations, 4 estimate lack of fit formulations and 4 replicates formulations were randomly arranged by Design Expert® 6.09 software. The formulations and manufacturing procedures for these fourteen placebo runs for CARBOPOL 974P and NOVEON AA-1 are described below.

TABLE 1
Experimental Design for Placebo Film Formulations
with CARBOPOL 974P

Levels

| Formulation Variables | Low (%) | High (%) |
|---|---|---|
| X1: Level of PVA | 70 | 95 |
| X2: Level of Glycerin | 5 | 20 |
| X3: Level of CARBOPOL 974P | 0 | 10 |

Formulation and Manufacturing Process for Placebo Film with Carbopol 974P for Statistical Design

Run #C1

| Ingredients | mg/film | w/w % | g/batch |
|---|---|---|---|
| Polyvinyl Alcohol (87-89% partially | 285 | 95.00 | 14.25 |
| hydrolyzed MW 11000-31000) | | | |
| Glycerin | 15 | 5.00 | 0.75 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution of Step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 15 hours.

Run #C2

| Ingredients | mg/film | w/w % | g/batch |
|---|---|---|---|
| Polyvinyl Alcohol (87-89% partially | 210 | 70.00 | 10.5 |
| hydrolyzed MW 11000-31000) | | | |
| Glycerin | 60 | 20.00 | 3 |
| Carbopol 974P | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 72 g of solution into a 150×15 mm style Petri Dish.
6. Dry in an oven at 40° C. for 40 hours.

Run #C3

| Ingredients | mg/film | w/w % | g/batch |
|---|---|---|---|
| Polyvinyl Alcohol (87-89% partially | 240 | 80.00 | 12 |
| hydrolyzed MW 11000-31000) | | | |
| Glycerin | 60 | 20.00 | 3 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution of Step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 30 hours.

Run #C4

| Ingredients | mg/film | w/w % | g/batch |
|---|---|---|---|
| Polyvinyl Alcohol (87-89% partially | 255 | 85.00 | 12.75 |
| hydrolyzed MW 11000-31000) | | | |
| Glycerin | 15 | 5.00 | 0.75 |
| Carbopol 974P | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 72 g of solution into a 150×15 mm style Petri Dish.
6. Dry in an oven at 40° C. for 24 hours.

Run #C5

| Ingredients | mg/film | w/w % | g/batch |
|---|---|---|---|
| Polyvinyl Alcohol (87-89% partially | 210 | 70.00 | 10.5 |
| hydrolyzed MW 11000-31000) | | | |
| Glycerin | 60 | 20.00 | 3 |
| Carbopol 974P | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 72 g of solution into a 150×15 mm style Petri Dish.
6. Dry in an oven at 40° C. for 50 hours.

Run #C6

| Ingredients | mg/film | w/w % | g/batch |
|---|---|---|---|
| Polyvinyl Alcohol (87-89% partially | 270 | 90.00 | 13.5 |
| hydrolyzed MW 11000-31000) | | | |
| Glycerin | 15 | 5.00 | 0.75 |
| Carbopol 974P | 15 | 5.00 | 0.75 |
| Total | 300 | 100.00 | 15 |
| De-ionized Water | 5700 | | 285 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 36 g of solution into a 150×15 mm style Petri Dish.
6. Dry in an oven at 40° C. for 24 hours.

Run #C7

| Ingredients | mg/film | w/w % | g/batch |
|---|---|---|---|
| Polyvinyl Alcohol (87-89% partially | 258.75 | 86.25 | 12.9375 |
| hydrolyzed MW 11000-31000) | | | |
| Glycerin | 26.25 | 8.75 | 1.3125 |
| Carbopol 974P | 15 | 5.00 | 0.75 |
| Total | 300 | 100.00 | 15 |
| De-ionized Water | 5700 | | 285 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 36 g of solution into a 150×15 mm style Petri Dish.
6. Dry in an oven at 40° C. for 30 hours.

Run #C8

| Ingredients | mg/film | w/w % | g/batch |
|---|---|---|---|
| Polyvinyl Alcohol (87-89% partially | 240 | 80.00 | 12 |
| hydrolyzed MW 11000-31000) | | | |
| Glycerin | 60 | 20.00 | 3 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution in step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 24 hours.

Run #C9

| Ingredients | mg/film | w/w % | g/batch |
|---|---|---|---|
| Polyvinyl Alcohol (87-89% partially | 247.5 | 82.50 | 12.375 |
| hydrolyzed MW 11000-31000) | | | |
| Glycerin | 37.5 | 12.50 | 1.875 |
| Carbopol 974P | 15 | 5.00 | 0.75 |
| Total | 300 | 100.00 | 15 |
| De-ionized Water | 5700 | | 285 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 36 g of solution into a 150×15 mm style Petri Dish.
6. Dry in an oven at 40° C. for 27 hours.

Run #C10

| Ingredients | mg/film | w/w % | g/batch |
|---|---|---|---|
| Polyvinyl Alcohol (87-89% partially | 262.5 | 87.50 | 13.125 |
| hydrolyzed MW 11000-31000) | | | |
| Glycerin | 37.5 | 12.50 | 1.875 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution in step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 14 hours.

Run #C11

| Ingredients | mg/film | w/w % | g/batch |
|---|---|---|---|
| Polyvinyl Alcohol (87-89% partially | 225 | 75.00 | 11.25 |
| hydrolyzed MW 11000-31000) | | | |
| Glycerin | 60 | 20.00 | 3 |
| Carbopol 974P | 15 | 5.00 | 0.75 |
| Total | 300 | 100.00 | 15 |
| De-ionized Water | 5700 | | 285 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 36 g of solution into a 150×15 mm style Petri Dish.
6. Dry in an oven at 40° C. for 24 hours.

Run #C12

| Ingredients | mg/film | w/w % | g/batch |
|---|---|---|---|
| Polyvinyl Alcohol (87-89% partially | 285 | 95.00 | 14.25 |
| hydrolyzed MW 11000-31000) | | | |
| Glycerin | 15 | 5.00 | 0.75 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution in step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 15 hours.

Run #C13

| Ingredients | mg/film | w/w % | g/batch |
|---|---|---|---|
| Polyvinyl Alcohol (87-89% partially | 228.75 | 76.25 | 11.4375 |
| hydrolyzed MW 11000-31000) | | | |
| Glycerin | 48.75 | 16.25 | 2.4375 |
| Carbopol 974P | 22.5 | 7.50 | 1.125 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Carbopol 974P into the solution by continuous stirring.
4. Stand overnight to remove any bubbles.
5. Pour 72 g of solution into a 150×15 mm style Petri Dish.
6. Dry in an oven at 40° C. for 40 hours.

Run #C14

| Ingredients | mg/film | w/w % | g/batch |
|---|---|---|---|
| Polyvinyl Alcohol (87-89% partially | 255 | 85.00 | 12.75 |
| hydrolyzed MW 11000-31000) | | | |
| Glycerin | 15 | 5.00 | 0.75 |
| Carbopol 974P | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and remove the heat and dissolve Carbopol 974P into the solution by continuous stirring.
4. Stand overnight to remove any bubbles.
5. Pour 72 g of solution into a 150×15 mm style Petri Dish.
6. Dry in an oven at 40° C. for 24 hours.

TABLE 2
Experimental Design for Placebo Film Formulations with NOVEON AA-1

Levels

| Formulation Variables | Low (%) | High (%) |
|---|---|---|
| X1: Level of PVA | 70 | 95 |
| X2: Level of Glycerin | 5 | 20 |
| X3: Level of NOVEON AA-1 | 0 | 10 |

Formulation and Manufacturing Process for Placebo Film with Noveon® AA-1 for Statistical Design

Run #N1

| Ingredients | mg/film | w/w % | g/batch |
|---|---|---|---|
| Polyvinyl Alcohol (87-89% partially | 258.75 | 86.25 | 12.9375 |
| hydrolyzed MW 11000-31000) | | | |
| Glycerin | 26.25 | 8.75 | 1.3125 |
| Noveon ® AA-1 | 15 | 5.00 | 0.75 |
| Total | 300 | 100.00 | 15 |
| De-ionized Water | 5700 | | 285 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Noveon® AA-1 into the solution by continuous stirring.
4. Stand overnight to remove any bubbles.
5. Pour 36 g of solution into a 150×15 mm style Petri Dish.
6. Dry in an oven at 40° C. for 9 hours.

Run #N2

| Ingredients | mg/film | w/w % | g/batch |
|---|---|---|---|
| Polyvinyl Alcohol (87-89% partially | 228.75 | 76.25 | 11.4375 |
| hydrolyzed MW 11000-31000) | | | |
| Glycerin | 48.75 | 16.25 | 2.4375 |
| Noveon ® AA-1 | 22.5 | 7.50 | 1.125 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Noveon® AA-1 into the solution by continuous stirring.
4. Stand overnight to remove any bubbles.
5. Pour 72 g of solution into a 150×15 mm style Petri Dish.
6. Dry in an oven at 40° C. for 40 hours.

Run #N3

| Ingredients | mg/film | w/w % | g/batch |
|---|---|---|---|
| Polyvinyl Alcohol (87-89% partially | 210 | 70.00 | 10.5 |
| hydrolyzed MW 11000-31000) | | | |
| Glycerin | 60 | 20.00 | 3 |
| Noveon ® AA-1 | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Noveon® AA-1 into the solution by continuous stirring.
4. Stand for overnight to remove any bubbles.
5. Pour 72 g of solution into a 150×15 mm style Petri Dish.
6. Dry in an oven at 40° C. for 25 hours.

Run #N4

| Ingredients | mg/film | w/w % | g/batch |
|---|---|---|---|
| Polyvinyl Alcohol (87-89% partially | 285 | 95.00 | 14.25 |
| hydrolyzed MW 11000-31000) | | | |
| Glycerin | 15 | 5.00 | 0.75 |
| Total | 300 | 100.00 | 15 |
| De-ionized Water | 2700 | | 135 |

### Procedures:

1. Dissolve Glycerin into the De-ionized Water at RT.
2. Dissolve PVA into the same solution in step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 15 hours.

Run #N5

| Ingredients | mg/film | w/w % | g/batch |
|---|---|---|---|
| Polyvinyl Alcohol (87-89% partially | 232.5 | 77.50 | 11.625 |
| hydrolyzed MW 11000-31000) | | | |
| Glycerin | 37.5 | 12.50 | 1.875 |
| Noveon ® AA-1 | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Noveon® AA-1 into the solution by continuous stirring.
4. Stand overnight to remove any bubbles.
5. Pour 72 g of solution into a 150×15 mm style Petri Dish.
6. Dry in an oven at 40° C. for 40 hours.

Run #N6

| Ingredients | mg/film | w/w % | g/batch |
|---|---|---|---|
| Polyvinyl Alcohol (87-89% partially | 285 | 95.00 | 14.25 |
| hydrolyzed MW 11000-31000) | | | |
| Glycerin | 15 | 5.00 | 0.75 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution in step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 15 hours.

Run #N7

| Ingredients | mg/film | w/w % | g/batch |
|---|---|---|---|
| Polyvinyl Alcohol (87-89% partially | 270 | 90.00 | 13.5 |
| hydrolyzed MW 11000-31000) | | | |
| Glycerin | 15 | 5.00 | 0.75 |
| Noveon ® AA-1 | 15 | 5.00 | 0.75 |
| Total | 300 | 100.00 | 15 |
| De-ionized Water | 5700 | | 285 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Noveon® AA-1 into the solution by continuous stirring.
4. Stand overnight to remove any bubbles.
5. Pour 36 g of solution into a 150×15 mm style Petri Dish.
6. Dry in an oven at 40° C. for 15 hours.

Run #N8

| Ingredients | mg/film | w/w % | g/batch |
|---|---|---|---|
| Polyvinyl Alcohol (87-89% partially | 240 | 80.00 | 12 |
| hydrolyzed MW 11000-31000) | | | |
| Glycerin | 60 | 20.00 | 3 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution in step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 30 hours.

Run #N9

| Ingredients | mg/film | w/w % | g/batch |
|---|---|---|---|
| Polyvinyl Alcohol (87-89% partially | 262.5 | 87.50 | 13.125 |
| hydrolyzed MW 11000-31000) | | | |
| Glycerin | 37.5 | 12.50 | 1.875 |
| Total | 300 | 100.00 | 15 |
| DI Water | 2700 | | 135 |

### Procedures:

1. Dissolve Glycerin into the DI Water at RT.
2. Dissolve PVA into the same solution in step 1 by heating the solution to NMT 75 C. and stirring.
3. Stand for overnight to remove any bubbles.
4. Pour 18 g of solution to a 150×15 mm style Petri Dish.
5. Dry in an oven at 40° C. for 14 hours.

Run #N10

| Ingredients | mg/film | w/w % | g/batch |
|---|---|---|---|
| Polyvinyl Alcohol (87-89% partially | 210 | 70.00 | 10.5 |
| hydrolyzed MW 11000-31000) | | | |
| Glycerin | 60 | 20.00 | 3 |
| Noveon ® AA-1 | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Noveon® AA-1 into the solution by continuous stirring.
4. Stand overnight to remove any bubbles.
5. Pour 72 g of solution into a 150×15 mm style Petri Dish.
6. Dry in an oven at 40° C. for 40.08 hours.

Run #N11

| Ingredients | mg/film | w/w % | g/batch |
|---|---|---|---|
| Polyvinyl Alcohol (87-89% partially | 247.5 | 82.50 | 12.375 |
| hydrolyzed MW 11000-31000) | | | |
| Glycerin | 37.5 | 12.50 | 1.875 |
| Noveon ® AA-1 | 15 | 5.00 | 0.75 |
| Total | 300 | 100.00 | 15 |
| De-ionized Water | 5700 | | 285 |

### Procedures:

1. Dissolve Glycerin into the De-ionized Water at RT.
2. Dissolve PVA into the same solution in step 1 by heating the solution to NMT 75° C. and stirring.
3. Remove the heat and dissolve Noveon® AA-1 into the previous solution by continuous stirring.
4. Stand overnight to remove any bubbles.
5. Pour 36 g of solution into a 150×15 mm style Petri Dish.
6. Dry in an oven at 40° C. for 9 hours.

Run #N12

| Ingredients | mg/film | w/w % | g/batch |
|---|---|---|---|
| Polyvinyl Alcohol (87-89% partially | 255 | 85.00 | 12.75 |
| hydrolyzed MW 11000-31000) | | | |
| Glycerin | 15 | 5.00 | 0.75 |
| Noveon ® AA-1 | 30 | 10.00 | 1.5 |
| Total | 300 | 100.00 | 15 |
| De-ionized Water | 11700 | | 585 |

### Procedures:

1. Dissolve Glycerin into the De-ionized Water at RT.
2. Dissolve PVA into the same solution in step 1 by heating the solution to NMT 75° C. and stirring.
3. Remove the heat and dissolve Noveon® AA-1 into the solution by continuous stirring.
4. Stand overnight to remove any bubbles.
5. Pour 72 g of solution into a 150×15 mm style Petri Dish.
6. Dry in an oven at 40° C. for 15 hours.

Run #N13

| Ingredients | mg/film | w/w % | g/batch |
|---|---|---|---|
| Polyvinyl Alcohol (87-89% partially | 240 | 80.00 | 12 |
| hydrolyzed MW 11000-31000) | | | |
| Glycerin | 60 | 20.00 | 3 |
| Total | 300 | 100.00 | 15 |
| De-ionized Water | 2700 | | 135 |

### Procedures:

1. Dissolve Glycerin into the De-ionized Water at room temperature.
2. Dissolve PVA into the same solution in step 1 by heating the solution to NMT 75° C. and stirring.
3. Stand overnight to remove any bubbles.
4. Pour 18 g of solution into a 150×15 mm style Petri Dish.
5. Dry in an oven at 40° C. for 24 hours.

Run #N14

| Ingredients | mg/film | w/w % | g/batch |
|---|---|---|---|
| Polyvinyl Alcohol (87-89% partially | 255 | 85.00 | 12.75 |
| hydrolyzed MW 11000-31000) | | | |
| Glycerin | 15 | 5.00 | 0.75 |
| Noveon ® AA-1 | 30 | 10.00 | 1.5 |
| Total | 300 | 100.00 | 15 |
| De-ionized Water | 11700 | | 585 |

### Procedures:

1. Dissolve Glycerin into the De-ionized Water at room temperature.
2. Dissolve PVA into the same solution in step 1 by heating the solution to NMT 75° C. and stirring.
3. Remove the heat and dissolve Noveon® AA-1 into the solution by continuous stirring.
4. Stand overnight to remove any bubbles.
5. Pour 72 g of solution into a 150×15 mm style Petri Dish.
6. Dry in an oven at 40° C. for 24 hours.

### Mechanical Properties of the Film: Tensile Strength, Percent Elongation, and Elastic Modulus

Physico-mechanical properties of the film were tested using a TA.XTPlus Texture Analyzer (Texture Technologies) equipped with a 50 kg load cell and TA-96 grips. Texture Exponent™ software was utilized for this purpose. The film was placed between the grips in order to achieve a sample size equivalent to approximately 15×25 mm². The lower grip was fixed and the upper grip was moved upward at the rate of 5 mm/s while data acquisition was processing and terminated when the film failed.

The tensile strength, percent elongation, and elastic modulus were used as indicators of the mechanical properties of the films. The tensile strength—strain curve was recorded for samples and ultimate tensile strength (force per unit cross sectional area required to break the film) determined. The maximum tensile strength is the largest stress that a film is able to sustain. The percent elongation to break the film is the maximum percent change in the length of film before breaking. The elastic modulus (slope of linear portion of curve) measures the resistance of the material to small deformations and is a measure of the stiffness (or rigidity) of the material. For each test, three determinations were carried out.

### Visual Dissolution of Placebo Film

The visual dissolution was performed using USP Apparatus 2, at 100 rpm in 900 mL of pH 4.5 acetate buffer under 37° C. The film sample of approximately 17×37 mm was placed into a capsule sinker (1.0″ length, 0.575″ I.D.) and dropped into the bottom of vessel. The time taken for the film sample to completely dissolve, which is defined that no piece of film is left by visual observation, was observed. All the tests were performed by the same individual. For each film formulation, three determinations were measured.

### Bioadhesive Properties of Placebo Films

Adhesion studies were performed on the films using following method. A film sample of a round shape with approximately 23 mm diameter was taped onto a metal ring with double-sided tape. The diameter of the ring was approximately 23 mm. The center of the ring was hollow which had a diameter of approximately 8.5 mm. The weight of the ring was approximately 20 g. The ring with the film was placed towards the outer edge of a petri dish, which contained agar/mucin (1.1 %/2.2%) gel. The film was placed in contact with the gel with the pertri dish in the horizontal position. Another approximately 20 g of weight was placed on the top of the ring for 2 minutes. The extra 20 g of weight was removed. One side of the petri dish was raised to create an angle of 22°. The ring was at the top position. This will allow the appropriate angle for the ring to slide down. The time for the ring to slide down 150 mm was measured. The adhesion force between the film and agar/mucin surface was considered to be inversely related to the time needed for the ring to travel the predetermined distance. Three determinations were carried out.

### Formulation and Manufacturing Process for Conjugated Estrogens Film

The formulations of the CE film were designed based on the results from the experimentally designed runs of the placebo films. The results of the placebo experimental runs indicate that CARBOPOL974P and NOVEONAA-1 had a similar influence on mechanical properties, holding time, as well as visual dissolution time of films. Therefore, only CARBOPOL974P was evaluated for the films with Conjugated Estrogens, similar results are expected from NOVEON and other formulations in accordance with the description herein. From the placebo experimental design runs, it was also observed that formulations containing 10% CARBOPOL974P or NOVEONAA-1 took longer time to dissolve. Therefore, films with Conjugated Estrogens were formulated to contain CARBOPOL974P 7.5% as the highest concentration. Formulations are listed in Tables 3 to 6.

TABLE 3
Formulation A of Conjugated Estrogens Film with CARBOPOL974P
Batch #: L34575-69

| Ingredients | mg/film | w/w % | g/batch |
|---|---|---|---|
| Polyvinyl Alcohol (87-89% | 228.3 | 76.1 | 11.415 |
| partially hydrolyzed MW | | | |
| 11000-31000) | | | |
| Glycerin | 48.75 | 16.25 | 2.4375 |
| CARBOPOL974P | 22.5 | 7.5 | 1.125 |
| CE Concentrate @ 16 mg/g | 28.125 | 0.15 | 1.4063 |
| | (0.45 mg CE) | | (0.0225 g CE) |
| Total Solid | 300 | 100 | 15 |
| Purified Water | 11672.325 | NA | 583.6162 |

TABLE 4
Formulation B of Conjugated Estrogens Film with CARBOPOL974P
Batch #: L34575-71

| Ingredients | mg/film | w/w % | g/batch |
|---|---|---|---|
| Polyvinyl Alcohol (87-89% | 217.05 | 72.35 | 10.8525 |
| partially hydrolyzed | | | |
| MW 11000-31000) | | | |
| Glycerin | 60 | 20 | 3 |
| CARBOPOL974P | 22.5 | 7.5 | 1.125 |
| CE Concentrate @ 16 mg/g | 28.125 | 0.15 | 1.4063 |
| | (0.45 mg CE) | | (0.0225 g CE) |
| Total Solid | 300 | 100 | 15 |
| Purified Water | 11672.325 | NA | 583.6162 |

TABLE 5
Formulation C of Conjugated Estrogens Film with CARBOPOL974P
Batch #: L34575-74

| Ingredients | mg/film | w/w % | g/batch |
|---|---|---|---|
| Polyvinyl Alcohol (87-89% | 254.55 | 84.85 | 12.7275 |
| partially hydrolyzed | | | |
| MW 11000-31000) | | | |
| Glycerin | 30 | 10 | 1.5 |
| CARBOPOL974P | 15 | 5 | 0.75 |
| CE Concentrate @ 16 mg/g | 28.125 | 0.15 | 1.4063 |
| | (0.45 mg CE) | | (0.0225 g CE) |
| Total Solid | 300 | 100 | 15 |
| Purified Water | 11672.325 | NA | 583.6162 |

TABLE 6
Formulation D of Conjugated Estrogens Film
Batch #: L34575-75

| Ingredients | mg/film | w/w % | g/batch |
|---|---|---|---|
| Polyvinyl Alcohol (87-89% | 284.55 | 94.85 | 14.2275 |
| partially hydrolyzed | | | |
| MW 11000-31000) | | | |
| Glycerin | 15 | 5 | 0.75 |
| CE Concentrate @ 16 mg/g | 28.125 | 0.15 | 1.4063 |
| | (0.45 mg CE) | | (0.0225 g CE) |
| Total Solid | 300 | 100 | 15 |
| Purified Water | 11672.325 | NA | 583.6162 |
| Formatting | | | |

The films were prepared as following:
1. Dissolve the glycerin into the purified water at room temperature.
2. Dissolve PVA into the same solution in step 1 by heating the solution to not more than 75° C. and stirring. During stirring, the speed of the stirring bar is adjusted until the solution starts to have a vortex and solution is complete.
3. Remove the heat and dissolve CARBOPOL974P into the solution of step 2 by continuous stirring until the solution is clear. During stirring, the speed of the stirring bar is adjusted until the solution starts to have a vortex and solution is complete.
4. When the solution at Step 3 reaches room temperature, add the CE Concentrate and mix until completely dissolved under the same mixing condition as in Step 3 (approximately 1-2 hours).
5. Ultrasonicate the solution to remove any air bubbles using a Branson 3200 ultrasonicator for 0.5 to 3 hours. Sonication is stopped when no air bubbles are visible.
6. Dry (in a portable oven drier) suitable quantities of solutions in a stainless steel container at 40° C.±3° C. to get CE films with approximately 0.14 mm thickness.

### Content Uniformity of CE

The content uniformity of CE was determined on a sample size of 10 films of approximately 300 mg using USP method for Conjugated Estrogens.

### pH Adjustment of Conjugated Estrogens Films

The pH of the CE film solution before drying was adjusted using Tris(hydroxymethyl)-aminomethane (Tris) or Triethanolamine (TEA) to approximately pH 7.4. The formulations (Formulation #1 to #8) are displayed in Tables 7 to 14. The manufacturing process is described in the following paragraph.

TABLE 7
Formulation #1 of Conjugated Estrogens Film with CARBOPOL974P
pH Adjusted Using Tris (hydroxymethyl)-aminomethane
Batch # L34419-79-1

| Ingredients | mg/film | w/w % | g/batch |
|---|---|---|---|
| Polyvinyl Alcohol (87-89% | 228.3 | 68.81 | 11.415 |
| partially hydrolyzed | | | |
| MW 11000-31000) | | | |
| Glycerin | 48.75 | 14.69 | 2.4375 |
| CARBOPOL 974P | 22.5 | 6.78 | 1.125 |
| CE Concentrate @ 16 mg/g | 28.125 | 0.14 | 1.4063 |
| | (0.45 mg CE) | | (0.0225 g CE) |
| Tris | 31.78 | 9.58 | 1.5892 |
| Total Solid | 331.78 | 100.00 | 16.59 |
| Purified Water | NA | NA | 883.8 |

TABLE 8
Formulation #2 of Conjugated Estrogens Film with CARBOPOL974P
pH Adjusted Using Tris (hydroxymethyl)-aminomethane
Batch # L34419-79-2

| Ingredients | mg/film | w/w % | g/batch |
|---|---|---|---|
| Polyvinyl Alcohol (87-89% | 217.05 | 65.03 | 10.8525 |
| partially hydrolyzed | | | |
| MW 11000-31000) | | | |
| Glycerin | 60 | 17.98 | 3 |
| CARBOPOL 974P | 22.5 | 6.74 | 1.125 |
| CE Concentrate @ 16 mg/g | 28.125 | 0.13 | 1.4063 |
| | (0.45 mg CE) | | (0.0225 g CE) |
| Tris | 33.78 | 10.12 | 1.6892 |
| Total Solid | 333.78 | 100.00 | 16.69 |
| Purified Water | NA | NA | 883.8 |

TABLE 9
Formulation #3 of Conjugated Estrogens Film with CARBOPOL974P
pH Adjusted Using Tris (hydroxymethyl)-aminomethane
Batch # L34419-79-3

| Ingredients | mg/film | w/w % | g/batch |
|---|---|---|---|
| Polyvinyl Alcohol (87-89% | 254.55 | 79.90 | 12.7275 |
| partially hydrolyzed | | | |
| MW 11000-31000) | | | |
| Glycerin | 30 | 9.42 | 1.5 |
| CARBOPOL 974P | 15 | 4.71 | 0.75 |
| CE Concentrate @ 16 mg/g | 28.125 | 0.14 | 1.4063 |
| | (0.45 mg CE) | | (0.0225 g CE) |
| Tris | 18.59 | 5.84 | 0.9296 |
| Total Solid | 318.59 | 100.01 | 15.93 |
| Purified Water | NA | NA | 584.0 |

TABLE 10
Formulation #4 of Conjugated Estrogens Film
pH Adjusted Using Tris (hydroxymethyl)-aminomethane
Batch # L34419-79-4

| Ingredients | mg/film | w/w % | g/batch |
|---|---|---|---|
| Polyvinyl Alcohol (87-89% | 284.55 | 94.52 | 14.2275 |
| partially hydrolyzed | | | |
| MW 11000-31000) | | | |
| Glycerin | 15 | 4.98 | 0.75 |
| CE Concentrate @ 16 mg/g | 28.125 | 0.15 | 1.4063 |
| | (0.45 mg CE) | | (0.0225 g CE) |
| Tris | 1.05 | 0.35 | 0.0525 |
| Total Solid | 301.05 | 100.00 | 15.0525 |
| Purified Water | NA | NA | 584.0 |

TABLE 11
Formulation #5 of Conjugated Estrogens Film with CARBOPOL974P
pH Adjusted Using Triethanolamine
Batch # L34419-79-5

| Ingredients | mg/film | w/w % | g/batch |
|---|---|---|---|
| Polyvinyl Alcohol (87-89% | 228.3 | 68.89 | 11.415 |
| partially hydrolyzed | | | |
| MW 11000-31000) | | | |
| Glycerin | 48.75 | 14.71 | 2.4375 |
| CARBOPOL 974P | 22.5 | 6.79 | 1.125 |
| CE Concentrate @ 16 mg/g | 28.125 | 0.14 | 1.4063 |
| | (0.45 mg CE) | | (0.0225 g CE) |
| Triethanolamine (TEA) | 31.4 | 9.48 | 1.57 |
| Total Solid | 331.4 | 100.01 | 16.57 |
| Purified Water | NA | NA | 884 |

TABLE12
Formulation #6 of Conjugated Estrogens Film with CARBOPOL974P
pH Adjusted Using Triethanolamine
Batch # L34419-79-6

| Ingredients | mg/film | w/w % | g/batch |
|---|---|---|---|
| Polyvinyl Alcohol (87-89% | 217.05 | 65.31 | 10.8525 |
| partially hydrolyzed | | | |
| MW 11000-31000) | | | |
| Glycerin | 60 | 18.05 | 3 |
| CARBOPOL 974P | 22.5 | 6.77 | 1.125 |
| CE Concentrate @ 16 mg/g | 28.125 | 0.14 | 1.4063 |
| | (0.45 mg CE) | | (0.0225 g CE) |
| Triethanolamine (TEA) | 32.36 | 9.74 | 1.618 |
| Total Solid | 332.36 | 100.01 | 16.618 |
| Purified Water | NA | NA | 884 |

TABLE 13
Formulation #7 of Conjugated Estrogens Film with CARBOPOL974P
pH Adjusted Using Triethanolamine
Batch # L34419-79-7

| Ingredients | mg/film | w/w % | g/batch |
|---|---|---|---|
| Polyvinyl Alcohol (87-89% | 254.55 | 78.83 | 12.7275 |
| partially hydrolyzed | | | |
| MW 11000-31000) | | | |
| Glycerin | 30 | 9.29 | 1.5 |
| CARBOPOL 974P | 15 | 4.65 | 0.75 |
| CE Concentrate @ 16 mg/g | 28.125 | 0.14 | 1.4063 |
| | (0.45 mg CE) | | (0.0225 g CE) |
| Triethanolamine (TEA) | 22.93 | 7.10 | 1.1463 |
| Total Solid | 322.93 | 100.01 | 16.1463 |
| Purified Water | NA | NA | 584 |

TABLE 14
Formulation #8 of Conjugated Estrogens Film
pH Adjusted Using Triethanolamine
Batch # L34419-79-8

| Ingredients | mg/film | w/w % | g/batch |
|---|---|---|---|
| Polyvinyl Alcohol (87-89% | 284.55 | 94.24 | 14.2275 |
| partially hydrolyzed | | | |
| MW 11000-31000) | | | |
| Glycerin | 15 | 4.97 | 0.75 |
| CE Concentrate @ 16 mg/g | 28.125 | 0.15 | 1.4063 |
| | (0.45 mg CE) | | (0.0225 g CE) |
| Triethanolamine (TEA) | 1.93 | 0.64 | 0.0967 |
| Total Solid | 301.93 | 100.00 | 15.097 |
| Purified Water | NA | NA | 584.0 |

The procedure to make films of Formulations #1 to 8 is as following:
1. Dissolve glycerin into the purified water by stirring at room temperature.
2. Dissolve PVA into the same solution in step 1 by heating the solution to NMT 75° C. and stirring. During stirring, the speed of the stirring bar is adjusted until the solution starts to have a vortex and solution is complete.
3. Remove the heat and dissolve CARBOPOL 974P into the solution by continuous stirring. Measure the pH of above solution. During stirring, the speed of the stirring bar is adjusted until the solution starts to have a vortex and solution is complete.
4. Add either Tris or TEA into the above solution and stir until completely dissolved. Measure the pH of this solution. During stirring, the speed of the stirring bar is adjusted until the solution starts to have a vortex and solution is complete.
5. Add CE Concentrate and stir for 2-3 hours at room temperature.
6. Remove entrapped air bubble in the solution either by ultrasonication for 0.5 to 3 hours or by standing for 2-4 hours. No any bubbles existence by visual observation will be determined as the end of ultrasonication.
7. Dry suitable quantities of solutions in a stainless steel container at 40° C.±3° C. to get CE films with approximately 0.14 mm thickness.

### Dissolution of CE Films

The dissolution of CE from released from the film was determined by using USP Apparatus 2 at 50 rpm in 900 mL of pH 4.5 acetate buffer at 37° C. A film sample of approximately 40×40 mm was placed into a capsule sinker (31 mm length, 11 mm I.D.) and dropped into the bottom of vessel. The amount of CE released from the film was measured by high-performance liquid chromatography.

### Results and Discussion

### Data Analysis on Experimental Design Batches

### Mechanical Properties of Placebo Film With CARBOPOL 974P: Tensile Strength, Percent Elongation, and Elastic Modulus

The influence of levels of Glycerin, PVA and bioadhesive polymer, CARBOPOL 974P, on the mechanical properties of the film were evaluated using a D-optimal mixture experimental design. D-Optimal designs are designs which have trials selected to produce models that will provide the best estimates of the effects. D-Optimal designs are best used when screening factors. “D” stands for “Determinant.” D-Optimal designs provide the lowest determinant for the variance-covariance matrix for the effects. Mixture factor is a factor subject to the mixture constraint. This constraint requires that all ingredients in a mixture must add to 100%. For mixture designs, the trace plot shows the effects of changing each component along an imaginary line from the reference blend (defaulted to the overall centroid) to the vertex. As the amount of this component increases, the amounts of other component decreases, but their ratio to one another remains constant. On the trace plot, a steep slope or curvature in an input variable indicates a relatively high sensitivity of response. Table 15 displays the results of mechanical properties of the placebo films from experimental design runs using CARBOPOL 974P.

TABLE 15
Mechanical Properties of Placebo Film from Experimental Design Runs Using
Carbopol 974P As Bioadhesive Polymer

| | | | | | | Elastic |
|---|---|---|---|---|---|---|
| | | | | Tensile | | Modulus |
| | | | Carbopol | Strength | | mPa/% × |
| Run # | PVA % | Glycerin % | 974P % | (mPa) | Elongation % | 1000 |
| 1 | 95.00 | 5.00 | 0.00 | 0.29 ± 0.09 | 120 ± 5 | 22.29 ± 1.55 |
| 2 | 70.00 | 20.00 | 10.00 | 0.11 ± 0.02 | 475 ± 50 | 0.83 ± 0.12 |
| 3 | 80.00 | 20.00 | 0.00 | 0.14 ± 0.01 | 410 ± 31 | 1.15 ± 0.43 |
| 4 | 85.00 | 5.00 | 10.00 | 0.41 ± 0.06 | 111 ± 1 | 40.98 ± 4.55 |
| 5 | 70.00 | 20.00 | 10.00 | 0.16 ± 0.03 | 426 ± 63 | 1.7 ± 0.45 |
| 6 | 90.00 | 5.00 | 5.00 | 0.31 ± 0.12 | 237 ± 177 | 30.04 ± 1.7 |
| 7 | 86.25 | 8.75 | 5.00 | 0.23 ± 0.04 | 391 ± 46 | 5.38 ± 1.19 |
| 8 | 80.00 | 20.00 | 0.00 | 0.18 ± 0.02 | 606 ± 126 | 1.95 ± 0.41 |
| 9 | 82.50 | 12.50 | 5.00 | 0.18 ± 0.07 | 305 ± 106 | 4.13 ± 0.89 |
| 10 | 87.50 | 12.50 | 0.00 | 0.14 ± 0.04 | 450 ± 52 | 1.53 ± 0.47 |
| 11 | 75.00 | 20.00 | 5.00 | 0.18 ± 0.03 | 490 ± 68 | 1.6 ± 0.24 |
| 12 | 95.00 | 5.00 | 0.00 | 0.26 ± 0.11 | 122 ± 4 | 14.03 ± 3.53 |
| 13 | 76.25 | 16.25 | 7.50 | 0.18 ± 0.02 | 409 ± 43 | 1.97 ± 0.33 |
| 14 | 85.00 | 5.00 | 10.00 | 0.38 ± 0.06 | 110 ± 1 | 36.5 ± 6.7 |

Tensile strength, percent elongation, and elastic modulus of all model formulations were treated by Design Expert 6.0.9 software. Suitable models for these experiments include linear, quadratic and special cubic models. The best fitting mathematical model was selected based on the comparisons of several statistical parameters including the standard deviation (ST), the multiple correlation coefficient (R²), adjusted multiple correlation coefficient (adjusted R²), predicted multiple correlation coefficient (Predicted R²), the predicted residual sum of square (PRESS), and adequate precision provided by Design Expert 6.09 software. Among these statistical parameters, PRESS indicates how well the model fits the data, and for the chosen model it should be small relative to the other models under consideration. The predicted R² is in reasonable agreement with the adjusted R². The adequate precision measures the signal to noise ratio. A ratio greater than 4 is desirable.

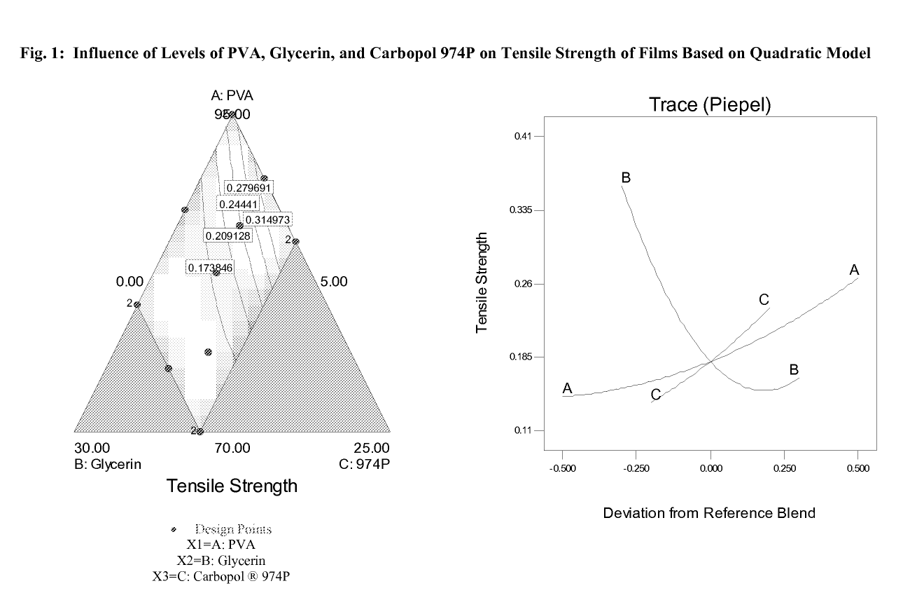

As shown in Table 16, the response values of tensile strength data were best fit by a quadratic model. In the case of the percent elongation, the linear model was most suitable. For elastic modulus, after natural log transformation, the best fit was the quadratic model. FIG. 1 shows that the increase levels of PVA and CARBOPOL 974P will increase the film tensile strength. The influence of CARBOPOL 974P on the tensile strength is greater than PVA (as percent weight basis). On the other hand, increasing the level of glycerin will decrease the film tensile strength.

TABLE 16
Optimal Regression Equation for Each Response Variable for
Mechanical Properties of Placebo Film from Experimental Design Runs
Using Carbopol ® 974P As Bioadhesive Polymer

| | | Tensile | | |
|---|---|---|---|---|
| | | Strength | | Ln of Elastic Modulus |
| Model | Coefficient | (mPa) | Elongation % | mPa/% × 1000 |
| Linear | Std. Dev. | 0.047 | 78.69 | 0.52 |
| | R-Square | 0.7764 | 0.8085 | 0.8839 |
| | Adjusted R-Square | 0.7358 | 0.7737 | 0.8628 |
| | Predicted R-Square | 0.6164 | 0.6940 | 0.8121 |
| | PRESS | 0.043 | 1.088E+005 | 4.87 |
| | Adeq. Precision | 10.608 | 10.532 | 14.058 |
| Quadratic | Std. Dev | 0.025 | 78.44 | 0.33 |
| | R-Square | 0.9542 | 0.8616 | 0.9671 |
| | Adjusted R-Square | 0.9256 | 0.7751 | 0.9465 |
| | Predicted R-Square | 0.8551 | 0.5824 | 0.9005 |
| | PRESS | 0.016 | 1.484E+005 | 2.58 |
| | Adeq. Precision | 14.774 | 7.464 | 15.976 |
| Special Cubic | Std. Dev. | 0.021 | 68.42 | 0.33 |
| | R-Square | 0.9711 | 0.9079 | 0.9712 |
| | Adjusted R-Square | 0.9463 | 0.8289 | 0.9465 |
| | Predicted R-Square | 0.8887 | 0.7138 | 0.8912 |
| | PRESS | 0.012 | 1.018E+005 | 2.82 |
| | Adeq. Precision | 17.316 | 8.227 | 15.227 |

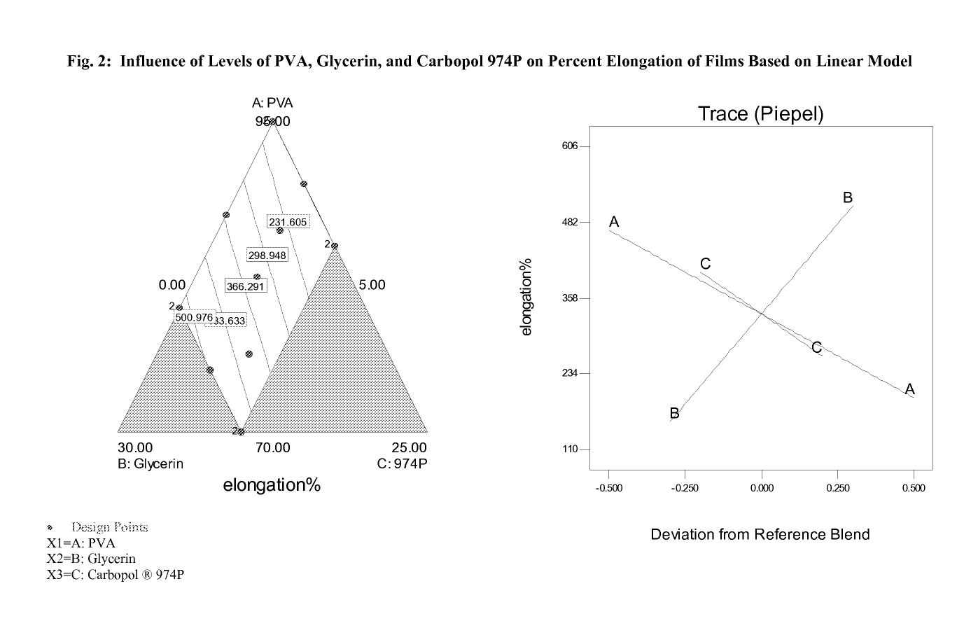

FIG. 2 indicates the influence of levels of these ingredients on elongation of film. From the figures it can be seen that glycerin will promote the elongation of the film; however, PVA and CARBOPOL 974P will have a negative effect on the film elongation.

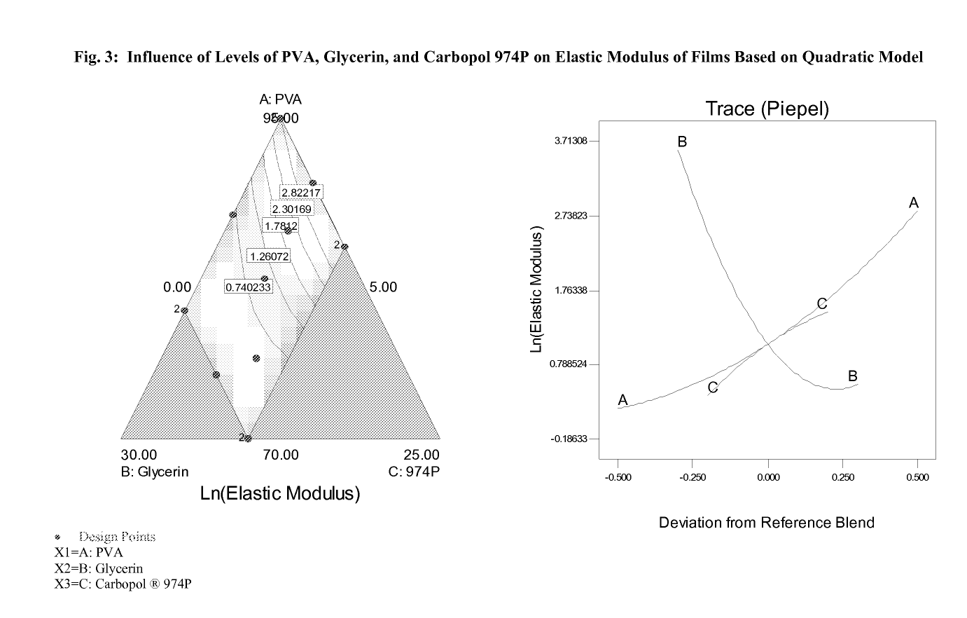

From FIG. 3 it can be concluded that increasing levels of PVA and CARBOPOL 974P will enhance the film elastic modulus, which indicates the film rigidity. However, the film toughness will be reduced when level of glycerin increases.

### Mechanical Properties of Placebo Film With NOVEON AA-1: Tensile Strength, Percent Elongation, and Elastic Modulus

Table 17 shows the results of mechanical properties of the placebo films from experimental design runs using NOVEON AA-1.

TABLE 17
Mechanical Properties of Placebo Film from Experimental Design Runs Using
Noveon AA-1 As Bioadhesive Polymer

| | | | | Tensile | | |
|---|---|---|---|---|---|---|
| | | | Noveon AA- | Strength | | Elastic Modulus |
| Run # | PVA % | Glycerin % | 1 % | (mPa) | Elongation % | mPa/% × 1000 |
| 1 | 86.25 | 8.75 | 5.00 | 0.28 ± 0.02 | 397 ± 30 | 6.96 ± 0.29 |
| 2 | 76.25 | 16.25 | 7.50 | 0.188 ± 0.06 | 404 ± 106 | 2.35 ± 0.33 |
| 3 | 70.00 | 20.00 | 10.00 | 0.138 ± 0.04 | 401 ± 87 | 0.94 ± 0.1 |
| 4 | 95.00 | 5.00 | 0.00 | 0.29 ± 0.09 | 120 ± 5 | 22.29 ± 1.55 |
| 5 | 77.50 | 12.50 | 10.00 | 0.234 ± 0.02 | 506 ± 148 | 2.14 ± 0.2 |
| 6 | 95.00 | 5.00 | 0.00 | 0.262 ± 0.11 | 122 ± 4 | 14.03 ± 3.53 |
| 7 | 90.00 | 5.00 | 5.00 | 0.264 ± 0.16 | 133 ± 8 | 13.31 ± 4.52 |
| 8 | 80.00 | 20.00 | 0.00 | 0.143 ± 0.01 | 410 ± 31 | 1.15 ± 0.43 |
| 9 | 87.50 | 12.50 | 0.00 | 0.139 ± 0.04 | 450 ± 52 | 1.53 ± 0.47 |
| 10 | 70.00 | 20.00 | 10.00 | 0.182 ± 0.02 | 522 ± 45 | 1.54 ± 0.18 |
| 11 | 82.50 | 12.50 | 5.00 | 0.221 ± 0.06 | 355 ± 71 | 5.13 ± 1.01 |
| 12 | 85.00 | 5.00 | 10.00 | 0.356 ± 0.09 | 124 ± 7 | 15.71 ± 1.11 |
| 13 | 80.00 | 20.00 | 0.00 | 0.185 ± 0.02 | 606 ± 126 | 1.95 ± 0.41 |
| 14 | 85.00 | 5.00 | 10.00 | 0.358 ± 0.02 | 123 ± 1 | 16.02 ± 1.01 |

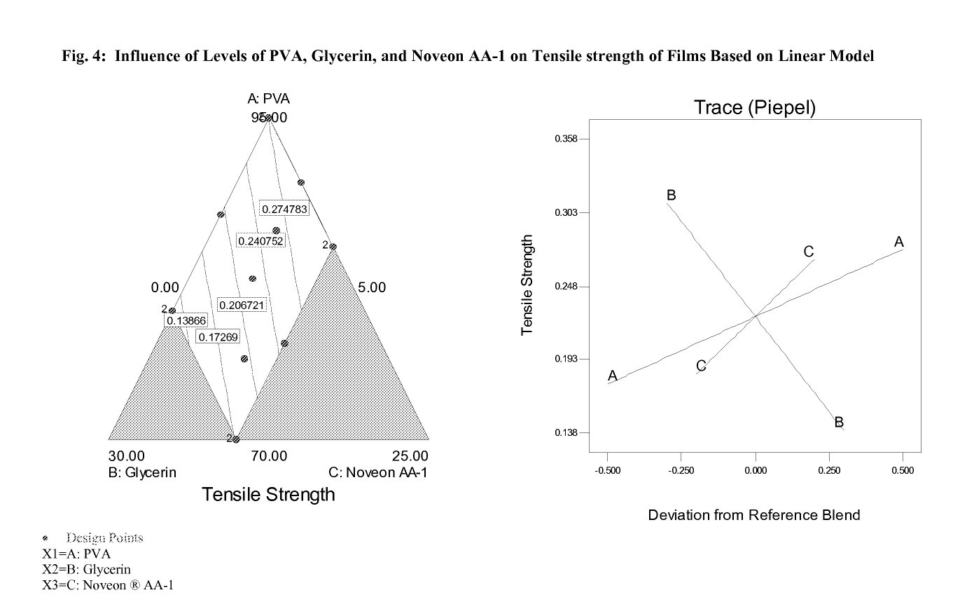

A statistical evaluation of the influence of NOVEON AA-1 on the mechanical properties of placebo film was carried out. As shown in Table 18, the approximations of response values of tensile strength and percent elongation were best described by a linear model. In the case of elastic modulus, after natural log transformation, it was the best fitted to the linear model. FIG. 4 shows that the increase levels of PVA and NOVEON AA-1 will increase the film tensile strength. And the effect of NOVEON AA-1 on the tensile strength is greater than PVA (as percent weight basis). On the other hand, increasing the level of glycerin will decrease the film tensile strength.

TABLE 18
Optimal Regression Equation for Each Response Variable for
Mechanical Properties of Placebo Film from Experimental Design
Runs Using Noveon AA-1 As Bioadhesive Polymer

| | | Tensile | | |
|---|---|---|---|---|
| | | Strength | | Ln of Elastic Modulus |
| Model | Coefficient | (mPa) | Elongation % | mPa/% × 1000 |
| Linear | Std. Dev. | 0.035 | 95.93 | 0.43 |
| | R-Square | 0.8096 | 0.7415 | 0.8775 |
| | Adjusted R-Square | 0.7750 | 0.6946 | 0.8552 |
| | Predicted R-Square | 0.6691 | 0.5813 | 0.8038 |
| | PRESS | 0.023 | 1.640E+005 | 3.28 |
| | Adeq. Precision | 12.190 | 8.115 | 12.746 |
| Quadratic | Std. Dev | 0.031 | 79.45 | 0.37 |
| | R-Square | 0.8922 | 0.8711 | 0.9344 |
| | Adjusted R-Square | 0.8248 | 0.7905 | 0.8935 |
| | Predicted R-Square | 0.6248 | 0.6123 | 0.7774 |
| | PRESS | 0.027 | 1.519E+005 | 3.72 |
| | Adeq. Precision | 9.703 | 7.456 | 11.596 |
| Special Cubic | Std. Dev. | 0.028 | 84.77 | 0.33 |
| | R-Square | 0.9222 | 0.8716 | 0.9536 |
| | Adjusted R-Square | 0.8556 | 0.7615 | 0.9139 |
| | Predicted R-Square | 0.4129 | 0.3716 | 0.7561 |
| | PRESS | 0.042 | 2.462E+005 | 4.07 |
| | Adeq. Precision | 10.081 | 6.390 | 11.925 |

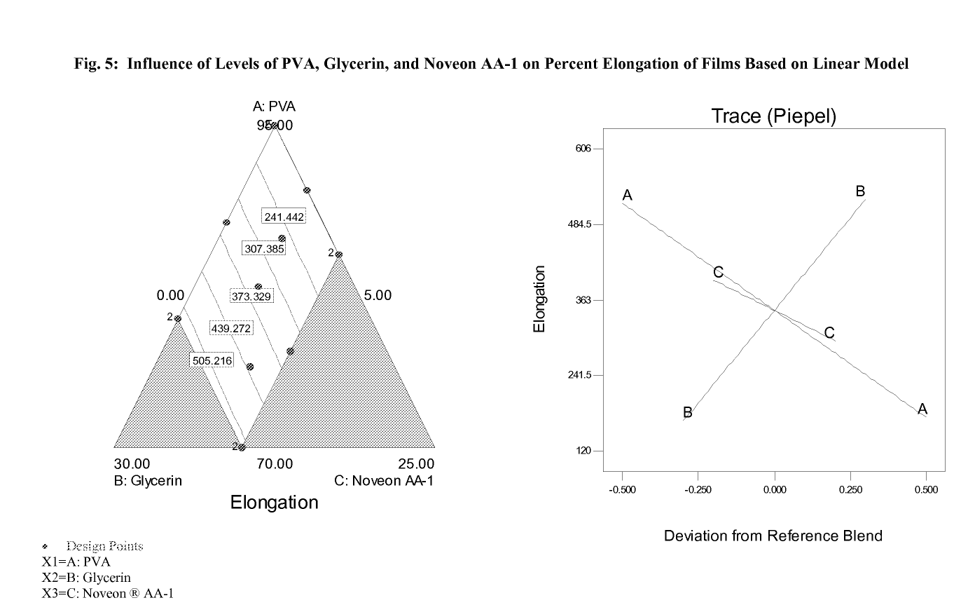

FIG. 5 indicates the influence of levels of these ingredients on elongation of film. From the figures it can be seen that glycerin will promote the elongation of the film; however, PVA and NOVEON AA-1 will have a negative effect on the film elongation.

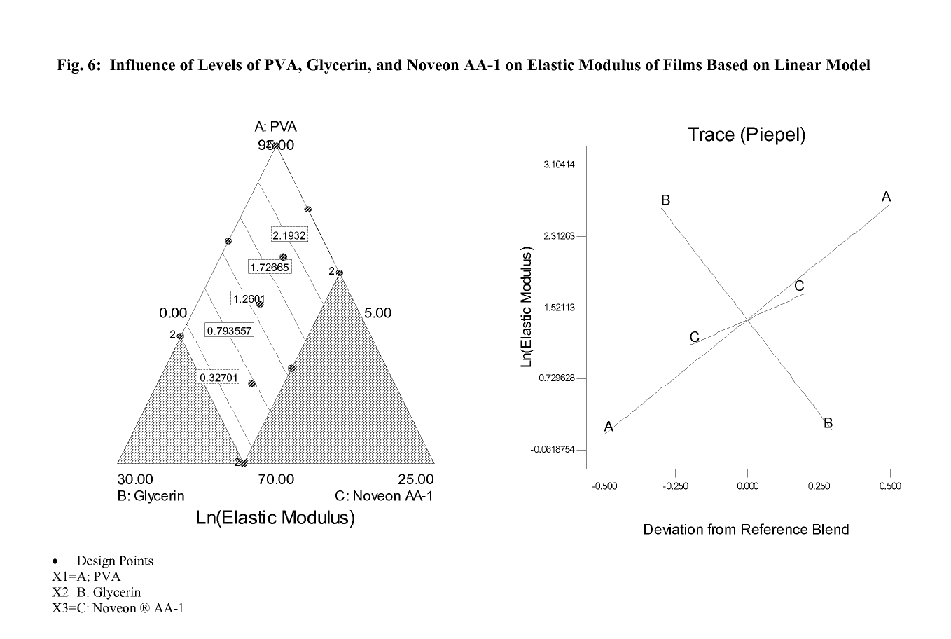

From FIG. 6 it can be concluded that increasing levels of PVA and NOVEON AA-1 will enhance the film elastic modulus, which indicates the film rigidity. However, the film toughness will be reduced when level of glycerin increases.
Visual Dissolution of Placebo Films with CARBOPOL 974P and NOVEONAA-1

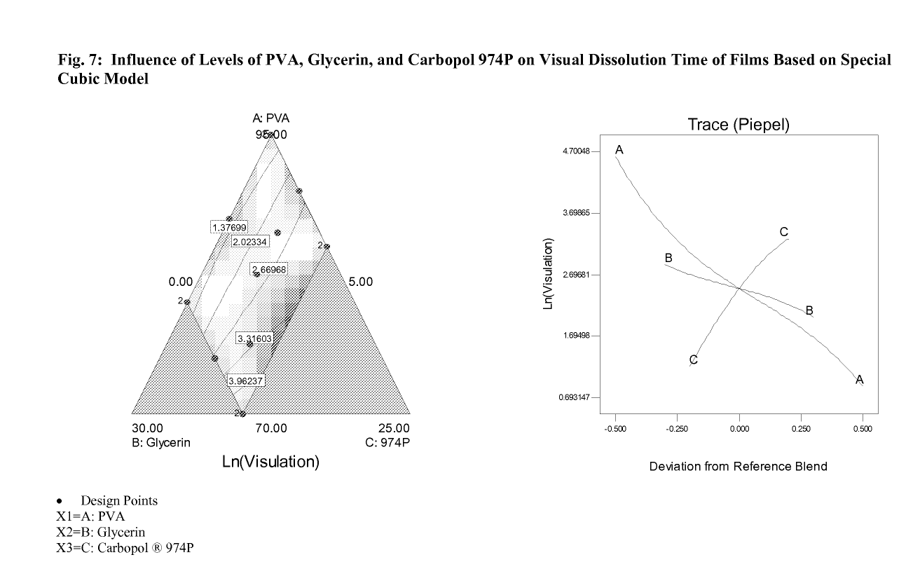

The influence of CARBOPOL 974P levels on the visual dissolution results is presented in Table 19. The statistical evaluation of the influence of CARBOPOL 974P on the visual dissolution of the placebo films is provided in Table 20 and FIG. 7. From the results it can be noted that the response values of visual dissolution of films with CARBOPOL 974P were best fit to the special cubic model after a natural log transformation. The results show that the increase levels of PVA and glycerin will increase the rate of dissolution of the film. On the other hand, increasing the level of CARBOPOL 974P will decrease the rate of dissolution of the film.

TABLE 19
Visual Dissolution Time of Placebo Film from Experimental
Design Runs Using Carbopol 974P As Bioadhesive Polymer

| | | | | Visual Dissolution |
|---|---|---|---|---|
| Run # | PVA % | Glycerin % | Carbopol 974P % | Time (min) |
| 1 | 95.00 | 5.00 | 0.00 | 3 ± 1.0 |
| 2 | 70.00 | 20.00 | 10.00 | 95.3 ± 9.5 |
| 3 | 80.00 | 20.00 | 0.00 | 3.3 ± 0.6 |
| 4 | 85.00 | 5.00 | 10.00 | 26.33 ± 2.1 |
| 5 | 70.00 | 20.00 | 10.00 | 110 ± 17.3 |
| 6 | 90.00 | 5.00 | 5.00 | 11.3 ± 0.6 |
| 7 | 86.25 | 8.75 | 5.00 | 12 ± 1.0 |
| 8 | 80.00 | 20.00 | 0.00 | 2.7 ± 0.6 |
| 9 | 82.50 | 12.50 | 5.00 | 12.7 ± 3.2 |
| 10 | 87.50 | 12.50 | 0.00 | 3 ± 1.7 |
| 11 | 75.00 | 20.00 | 5.00 | 14.33 ± 0.6 |
| 12 | 95.00 | 5.00 | 0.00 | 2 ± 0 |
| 13 | 76.25 | 16.25 | 7.50 | 25 ± 5.6 |
| 14 | 85.00 | 5.00 | 10.00 | 22.33 ± 1.5 |

TABLE 20
Optimal Regression Equation for Each Response Variable for Visual
Dissolution Time of Placebo Film from Experimental Design Runs
Using Carbopol 974P As Bioadhesive Polymer

| Model | Coefficient | Ln of Visual Dissolution Time |
|---|---|---|
| Linear | Std. Dev. | 0.32 |
| | R-Square | 0.9487 |
| | Adjusted R-Square | 0.9394 |
| | Predicted R-Square | 0.9056 |
| | PRESS | 2.05 |
| | Adeq. Precision | 24.129 |
| Quadratic | Std. Dev | 0.21 |
| | R-Square | 0.9837 |
| | Adjusted R-Square | 0.9735 |
| | Predicted R-Square | 0.9504 |
| | PRESS | 1.08 |
| | Adeq. Precision | 25.635 |
| Special Cubic | Std. Dev. | 0.15 |
| | R-Square | 0.9927 |
| | Adjusted R-Square | 0.9864 |
| | Predicted R-Square | 0.9688 |
| | PRESS | 0.68 |
| | Adeq. Precision | 34.918 |

Similarly, evaluation of the polymer levels on the visual dissolution of the placebo film with NOVEONAA-1 were also carried out. The results for these films are displayed in Table 21, 22 and FIG. 7, which provide the statistical analysis of the effect of levels of NOVEONAA-1 on the film visual dissolution. The response values of visual dissolution of films with NOVEON AA-1 were best fit by the quadratic model after the natural log transformation. From the results it can be noted that the increased level of PVA will help dissolution of the film. Alternatively by increasing the level of NOVEON AA-1 a decrease in the dissolution rate of the film is observed. In the case of glycerin, at the lower concentrations, it does not significantly affect the dissolution rate; however, at the higher levels, glycerin enhances the dissolution rate of films with NOVEON AA-1.

TABLE 21
Visual Dissolution Time of Placebo Film from Experimental Design
Runs Using NoveonAA-1 As Bioadhesive Polymer

| | | | | Visual Dissolution |
|---|---|---|---|---|
| Run # | PVA % | Glycerin % | Noveon AA-1 % | Time (min) |
| 1 | 86.25 | 8.75 | 5.00 | 22 ± 3.8 |
| 2 | 76.25 | 16.25 | 7.50 | 74 ± 13.5 |
| 3 | 70.00 | 20.00 | 10.00 | 89 ± 44.3 |
| 4 | 95.00 | 5.00 | 0.00 | 4 ± 0.6 |
| 5 | 77.50 | 12.50 | 10.00 | 70 ± 20 |
| 6 | 95.00 | 5.00 | 0.00 | 3 ± 0 |
| 7 | 90.00 | 5.00 | 5.00 | 19 ± 2.1 |
| 8 | 80.00 | 20.00 | 0.00 | 4 ± 0 |
| 9 | 87.50 | 12.50 | 0.00 | 5 ± 0.6 |
| 10 | 70.00 | 20.00 | 10.00 | 96 ± 30.7 |
| 11 | 82.50 | 12.50 | 5.00 | 28 ± 4.2 |
| 12 | 85.00 | 5.00 | 10.00 | 26 ± 4.2 |
| 13 | 80.00 | 20.00 | 0.00 | 4 ± 0.6 |
| 14 | 85.00 | 5.00 | 10.00 | 22 ± 4 |

TABLE 22
Optimal Regression Equation for Each Response Variable for Visual
Dissolution Time of Placebo Film from Experimental Design Runs
Using Noveon AA-1 As Bioadhesive Polymer

| Model | Coefficient | Ln of Visual Dissolution Time |
|---|---|---|
| Linear | Std. Dev. | 0.47 |
| | R-Square | 0.8879 |
| | Adjusted R-Square | 0.8675 |
| | Predicted R-Square | 0.8157 |
| | PRESS | 3.91 |
| | Adeq. Precision | 15.265 |
| Quadratic | Std. Dev | 0.13 |
| | R-Square | 0.9933 |
| | Adjusted R-Square | 0.9892 |
| | Predicted R-Square | 0.9807 |
| | PRESS | 0.41 |
| | Adeq. Precision | 38.11 |
| Special Cubic | Std. Dev. | 0.14 |
| | R-Square | 0.9936 |
| | Adjusted R-Square | 0.9881 |
| | Predicted R-Square | 0.9438 |
| | PRESS | 1.19 |
| | Adeq. Precision | 33.38 |

Bioadhesive Properties (Holding Time) of Placebo Films with CARBOPOL 974P and NOVEONAA-1

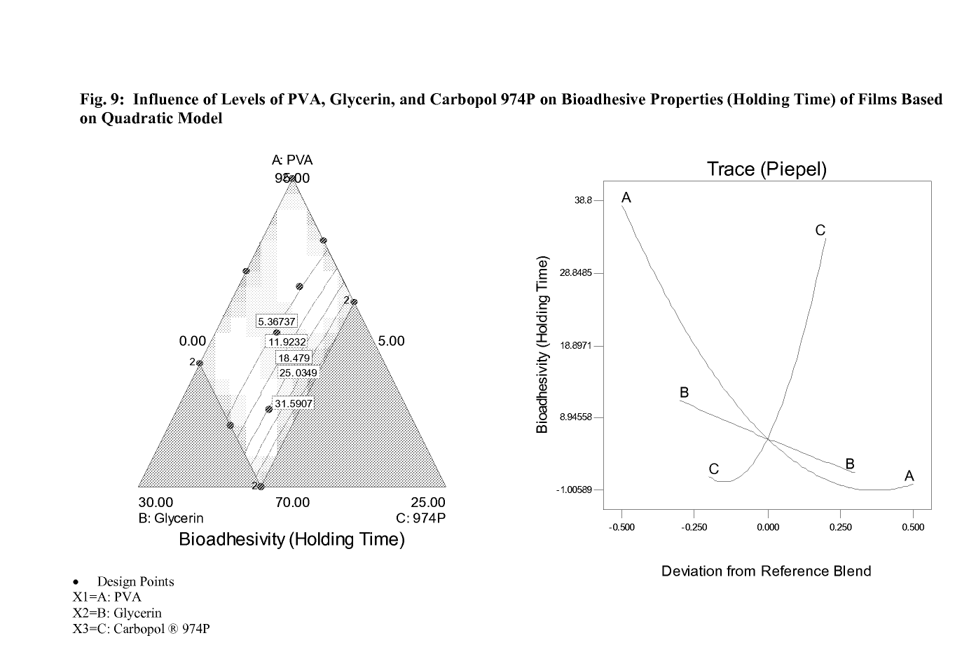

Table 23 displays the results of holding time, which is an indicator of the bioadhesive properties of the placebo films from the experimentally designed runs using CARBOPOL 974P. The statistical evaluation of the data for CARBOPOL 974P-containing film is provided in Table 24 and FIG. 9. The results indicate that the response values of bioadhesive properties of films with CARBOPOL 974P were best fit by the quadratic model. Increasing levels of PVA and glycerin will decrease the bioadhesive properties of the CARBOPOL 974P films. In addition the effect of PVA on the bioadhesive property is greater than glycerin. In contrast, increasing levels of CARBOPOL 974P will enhance the bioadhesive nature of the film.

TABLE 23
Bioadhesive Properties (Holding Time) of Placebo Film from
Experimental Design Runs Using Carbopol 974P As
Bioadhesive Polymer

| | | | | Holding Time |
|---|---|---|---|---|
| Run # | PVA % | Glycerin % | Carbopol 974P % | (min) |
| 1 | 95.00 | 5.00 | 0.00 | 0.25 ± 0.03 |
| 2 | 70.00 | 20.00 | 10.00 | 38.8 ± 9.3 |
| 3 | 80.00 | 20.00 | 0.00 | 0.34 ± 0.03 |
| 4 | 85.00 | 5.00 | 10.00 | 29.29 ± 10.4 |
| 5 | 70.00 | 20.00 | 10.00 | 36.2 ± 15.6 |
| 6 | 90.00 | 5.00 | 5.00 | 2.47 ± 0.61 |
| 7 | 86.25 | 8.75 | 5.00 | 4.3 ± 1.9 |
| 8 | 80.00 | 20.00 | 0.00 | 0.28 ± 0.03 |
| 9 | 82.50 | 12.50 | 5.00 | 4.8 ± 0.55 |
| 10 | 87.50 | 12.50 | 0.00 | 0.23 ± 0.05 |
| 11 | 75.00 | 20.00 | 5.00 | 7.25 ± 1.96 |
| 12 | 95.00 | 5.00 | 0.00 | 0.3 ± 0.03 |
| 13 | 76.25 | 16.25 | 7.50 | 22.1 ± 8.29 |
| 14 | 85.00 | 5.00 | 10.00 | 29.55 ± 16.01 |

TABLE 24
Optimal Regression Equation for Each Response Variable for
Bioadhesive Properties (Holding Time) of Placebo Film from
Experimental Design Runs Using Carbopol 974P As
Bioadhesive Polymer

| Model | Coefficient | Holding Time |
|---|---|---|
| Linear | Std. Dev. | 6.22 |
| | R-Square | 0.8541 |
| | Adjusted R-Square | 0.8276 |
| | Predicted R-Square | 0.7780 |
| | PRESS | 647.78 |
| | Adeq. Precision | 12.931 |
| Quadratic | Std. Dev | 1.47 |
| | R-Square | 0.9941 |
| | Adjusted R-Square | 0.9904 |
| | Predicted R-Square | 0.9857 |
| | PRESS | 41.67 |
| | Adeq. Precision | 39.77 |
| Special Cubic | Std. Dev. | 1.29 |
| | R-Square | 0.9960 |
| | Adjusted R-Square | 0.9926 |
| | Predicted R-Square | 0.9809 |
| | PRESS | 55.61 |
| | Adeq. Precision | 41.21 |

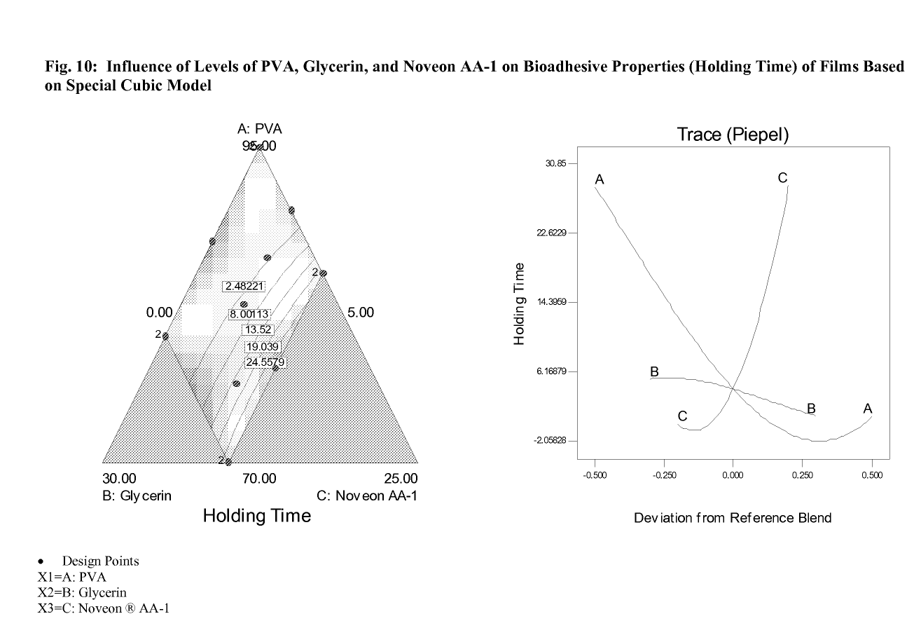

The influence of levels of NOVEONAA-1 on the bioadhesive nature of films with NOVEONAA-1 was also analyzed. Table 25 shows the results of holding times for these films. Table 26 and FIG. 10 indicate the response values of holding time of films with NOVEON AA-1 that were best fit by the special cubic model. As for CARBOPOL 974P films, increasing levels of PVA and glycerin decrease the bioadhesive properties of the NOVEON AA-1 films. The influence of PVA on bioadhesive properties is greater than glycerin. Increasing the level of NOVEON AA-1 will enhance the bioadhesive properties of the film.

TABLE 25
Bioadhesive Properties (Holding Time) of Placebo Film
from Experimental Design Runs Using NoveonAA-1
As Bioadhesive Polymer

| Run # | PVA % | Glycerin % | Noveon AA-1 % | Holding Time (min) |
|---|---|---|---|---|
| 1 | 86.25 | 8.75 | 5.00 | 0.94 ± 0.16 |
| 2 | 76.25 | 16.25 | 7.50 | 15.31 ± 1.64 |
| 3 | 70.00 | 20.00 | 10.00 | 27.91 ± 7.58 |
| 4 | 95.00 | 5.00 | 0.00 | 0.58 ± 0.13 |
| 5 | 77.50 | 12.50 | 10.00 | 30.85 ± 5.31 |
| 6 | 95.00 | 5.00 | 0.00 | 0.43 ± 0.08 |
| 7 | 90.00 | 5.00 | 5.00 | 0.75 ± 0.28 |
| 8 | 80.00 | 20.00 | 0.00 | 0.29 ± 0.02 |
| 9 | 87.50 | 12.50 | 0.00 | 0.37 ± 0.05 |
| 10 | 70.00 | 20.00 | 10.00 | 28.05 ± 6.15 |
| 11 | 82.50 | 12.50 | 5.00 | 2.97 ± 0.44 |
| 12 | 85.00 | 5.00 | 10.00 | 18.7 ± 2.97 |
| 13 | 80.00 | 20.00 | 0.00 | 0.24 ± 0.06 |
| 14 | 85.00 | 5.00 | 10.00 | 22.74 ± 3.31 |

TABLE 26
Optimal Regression Equation for Each Response Variable
for Bioadhesive Properties (Holding Time) of Placebo Film
from Experimental Design Runs Using Noveon AA-1 As
Bioadhesive Polymer

| Model | Coefficient | Holding Time |
|---|---|---|
| Linear | Std. Dev. | 5.41 |
| | R-Square | 0.8407 |
| | Adjusted R-Square | 0.8117 |
| | Predicted R-Square | 0.7713 |
| | PRESS | 461.91 |
| | Adeq. Precision | 12.32 |
| Quadratic | Std. Dev | 2.02 |
| | R-Square | 0.9838 |
| | Adjusted R-Square | 0.9737 |
| | Predicted R-Square | 0.9461 |
| | PRESS | 108.91 |
| | Adeq. Precision | 22.27 |
| Special Cubic | Std. Dev. | 1.59 |
| | R-Square | 0.9912 |
| | Adjusted R-Square | 0.9837 |

TABLE 25
Bioadhesive Properties (Holding Time) of Placebo Film
from Experimental Design Runs Using NoveonAA-1
As Bioadhesive Polymer

| | | | | |
|---|---|---|---|---|
| Run # | PVA % | Glycerin % | Noveon AA-1 % | Holding Time (min) |

| | |
|---|---|
| Predicted R-Square | 0.9507 |
| PRESS | 99.52 |
| Adeq. Precision | 27.35 |

### Content Uniformity of CE Films

Table 27 shows the results for the content uniformity for these formulations of CE films. The data indicate that the formulations and manufacture processes can yield CE films with excellent content uniformity.

TABLE 27
Content Uniformity of CE Films

| Formulation | Content | CU % |
|---|---|---|
| A | PVA/Glycerin/CARBOPOL 974P | 1.18 |
| | (76.1/16.25/7.5) | |
| B | PVA/Glycerin/CARBOPOL 974P | 2.10 |
| | (72.35/20/7.5) | |
| C | PVA/Glycerin/CARBOPOL 974P | 1.91 |
| | (84.85/10/5) | |
| D | PVA/Glycerin (94.85/5) | 1.42 |

### Influence of pH on Potency of CE Films

From the Table 28, it can be seen that the pH of the CE film solution has a big impact on the stability of the CE in the film after it has been dried. It was observed that the CE has an acceptable stability profile at a pH around 7.4. PH below than 5 caused low assay and high degradation results during the drying of the film. From this study it can also be seen that CE can tolerate 40° C. temperature for 48 hours as long as the pH of the CE film solution is more than 5. In contrast, adjusting the pH higher than 7.4 did not provide more benefit in terms of CE stability. For all formulations studied, no visual crystals on the films were noted after they were dried. The results also indicate that both Tris and TEA can serve as a stabilizer for CE by adjusting the pH of the solution to around 7.4.

TABLE 28
Assay Results of CE Films of Different Formulations

| | | CARBOPOL | Glycer- | | A- | |
|---|---|---|---|---|---|---|
| Batch # | pH | % | in % | TCE^{a} | DHEQN^{b} | EQN^{c} |
| CE Conc. | NA | NA | NA | 94.60 | 1.22 | 2.13 |
| Formulation A | 4.29 | 7.5 | 16.25 | 84.87 | 2.97 | 6.74 |
| Formulation B | 4.25 | 7.5 | 20 | 84.03 | 3.47 | 7.45 |
| Formulation C | 4.42 | 5 | 10 | 87.68 | 1.62 | 3.89 |
| Formulation D | 5.34 | 0 | 5 | 91.05 | 1.16 | 2.39 |
| Formulation #1 | 7.42 | 6.78 | 14.69 | 91.47 | 1.34 | 2.32 |
| Formulation #2 | 7.62 | 6.74 | 17.98 | 94.13 | 1.38 | 2.51 |
| Formulation #3 | 7.18 | 4.71 | 9.42 | 90.77 | 1.30 | 2.56 |
| Formulation #4 | 7.36 | 0 | 4.98 | 94.35 | 1.15 | 2.64 |
| Formulation #5 | 7.15 | 6.79 | 14.71 | 94.95 | 1.29 | 2.64 |
| Formulation #6 | 7.22 | 6.77 | 18.05 | 91.85 | 1.23 | 2.66 |
| Formulation #7 | 7.17 | 4.65 | 9.29 | 95.78 | 1.07 | 2.29 |
| Formulation #8 | 7.52 | 0 | 4.97 | 96.31 | 1.28 | 2.56 |

^{a}TCE (Total Conjugated Estrogens) includes Estrone, Equilin, and 17α-Dihydroequilin.
^{b}A-DHEQN is 17α-Dihydroequilenin.
^{c}EQN is Equilenin.

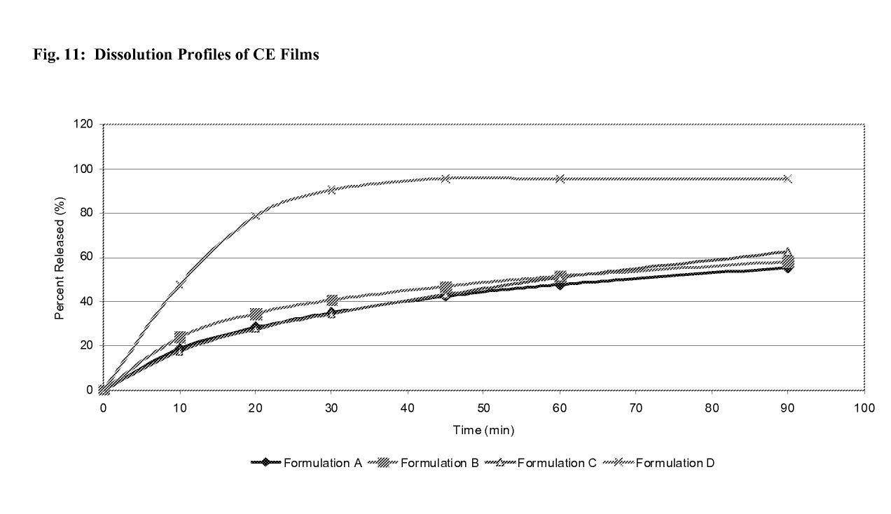

Dissolution Profiles of CE from Films
Dissolution profiles of CE from the films were measured. The effects of various levels of glycerin and polymers related to the dissolution rate of CE were evaluated in this study. The results are shown in Table 29 and FIG. 11. The results indicate that a similar trend to the visual dissolution was obtained. The addition of CARBOPOL 974P slowed down the rate of CE dissolution from the film. Increasing the level of glycerin increased the CE dissolution rate.

TABLE 29
Dissolution Results of CE from the Films

| Batch # (CARBOPOL | Dissolution (%) at Different Time (min) (% ± sd, n = 6) | | | | | | |
|---|---|---|---|---|---|---|---|
| 974P:Glycerin) | 0 | 10 | 20 | 30 | 45 | 60 | 90 |
| L34575-69 (7.5:16.25) | 0 | 19 ± 5.5 | 28 ± 6.3 | 35 ± 6.3 | 42 ± 5.3 | 48 ± 5.0 | 55 ± 5.8 |
| L34575-71 (7.5:20) | 0 | 24 ± 1.4 | 35 ± 1.0 | 41 ± 1.2 | 47 ± 1.8 | 51 ± 1.8 | 58 ± 1.7 |
| L34575-74 (5:10) | 0 | 18 ± 6.6 | 28 ± 9.4 | 35 ± 9.4 | 43 ± 8.7 | 51 ± 8.8 | 63 ± 10.4 |
| L34575-75 (0:5) | 0 | 48 ± 4.4 | 79 ± 7.5 | 90 ± 2.8 | 96 ± 2.0 | 96 ± 2.4 | 96 ± 2.5 |

### Influence of pH on Dissolution Profiles of CE Films

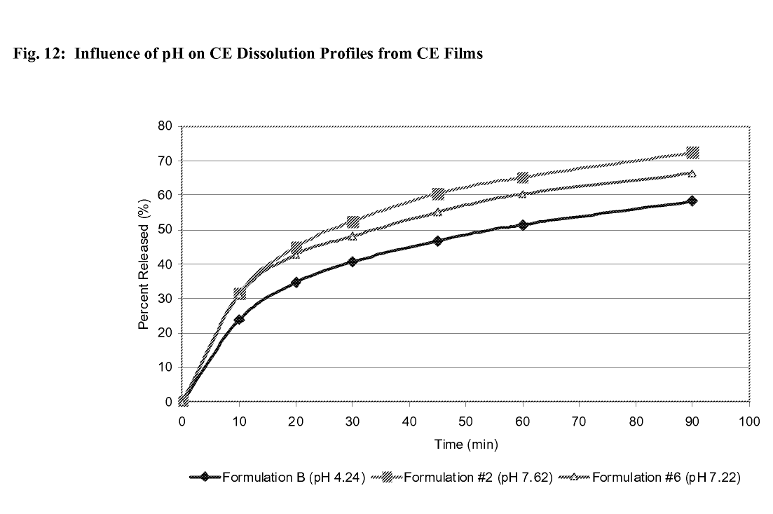

The dissolution profiles of CE from films of Formulation #2 and #6 were measured, respectively. Table 30 shows the results. FIG. 12 compares the dissolution profiles of CE before and after pH adjustment. The data indicate that the CE release is slightly increased after the pH adjustment.

TABLE 30
Influence of pH on CE Dissolution Profiles from CE Films

| Batch # | | | | | | | |
|---|---|---|---|---|---|---|---|
| (CARBOPOL | | | | | | | |
| 974P: | | | | | | | |
| Glycerin)/ | Dissolution (%) at Different Time (min) (% ± sd, n = 3) | | | | | | |
| pH Adjustor | 0 | 10 | 20 | 30 | 45 | 60 | 90 |
| L34575-71 | 0 | 24 ± 1.4 | 35 ± 1.0 | 41 ± 1.2 | 47 ± 1.8 | 51 ± 1.8 | 58 ± 1.7 |
| (7.5:20) | | | | | | | |
| L34419-79-2 | 0 | 32 ± 2.4 | 45 ± 0.8 | 52 ± 0.3 | 60 ± 0.9 | 65 ± 1.8 | 72 ± 1.0 |
| (7.5:20)/Tris | | | | | | | |
| L34419-79-6 | 0 | 31 ± 1.4 | 43 ± 1.7 | 48 ± 1.9 | 55 ± 1.9 | 60 ± 1.2 | 66 ± 2.1 |
| (7.5:20)/TEA | | | | | | | |

Thus, the bioadhesive film formulations and related manufacturing procedures are robust and proven to produce Conjugated Estrogens films. Changing levels of PVA, glycerin, and the bioadhesive polymer in the film can achieve different mechanical properties of the film. The level of bioadhesive polymer plays an important role for the bioadhesive properties of the film. As it is increased, the bioadhesive properties increase. The release rate of Conjugated Estrogens from the film can be influenced by the level of bioadhesive polymer. By adjusting various levels of the bioadhesive polymer, different and wide release rates can be achieved along with different film mechanical properties pending desired applications based on placebo studies described in this work. This novel delivery system can achieve the desired release characteristics for a CE delivery system while being robust and manufacturable.

The pH of the CE film solution plays an important role on the stability of the CE. CE can be stabilized by adjusting the pH with the use of a Tris or Tea buffer or equivalent of the solution to around 7.4.

In addition to the robust formulation aspects and multiple in-vitro characteristics, one can also conclude that one can achieve a range of in-vivo relationships in order to ascertain any combination within the ranges studied for a desired in-vivo effect. CE in-vivo responses covering a spectrum of relationships for a desired effect on individuals could be achieved within these formulation range studies.

The CE vaginal bioadhesive films described herein are for illustrative purposes only and are not meant to limit the invention in any way. As noted above, given the teachings herein, the film properties can be manipulated, particularly by altering the relative amounts of film forming agent, plasticizer, and adhesive agent in light of the film properties associated with a given active agent or combination of active agents.

What is claimed is:

1. A pharmaceutically acceptable bioadhesive film comprising:
About 65-95% film forming polymer by weight;
About 5-20% plasticizer by weight; and
Less than about 10% bioadhesive agent by weight;
and a pharmaceutically active agent.

2. The bioadhesive film of claim 1 wherein
said film forming polymer is selected from polyvinyl alcohol (PVA), polyethylene oxide, hydroxypropyl cellulose (HPC), hydroxyethyl cellulose (HEC), methylcellulose (MC), and hydroxypropyl methylcellulose (HPMC), copovidone, gelatin, maltodextrin, xanthan gum, guar gum, polymethacrylate, povidone, and mixtures thereof.

3. The bioadhesive film of claim 1, wherein
said plasticizer is selected from glycerin, polyethylene glycols, propylene glycol, mannitol, sorbitol, dibutyl phthalate, tributyl citrate, dimethyl phthalate, pyrrolidones, and mixtures thereof.

4. The bioadhesive film of claim 1, wherein
said bioadhesive agent is selected from polyacrylic acid derivatives, cellulose derivatives, substances of nature origin, protein, and mucilaginous substances from edible vegetables, and mixtures thereof.

5. The bioadhesive film of claim 1, wherein the bioadhesive agent is a polyacrylic acid derivative selected from high-molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, and combinations thereof.

6. The bioadhesive film of claim 1, wherein the bioadhesive agent is Carbomer Homopolymer Type B USP/NF(CARBOPOL 974P), and polycarbophil USP/NF (NOVEON AA-1).

7. The bioadhesive film of claim 1, wherein the bioadhesive agent is a cellulose derivative selected from alkylcelluloses, hydroxyalkylcelluloses, cellulose ethers, cellulose esters, nitrocelluloses, and combinations thereof.

8. The bioadhesive film of claim 1, wherein the bioadhesive is selected from substances of natural origin selected from chitosans, guar gum, xanthan gum, carrageenan, pectin, sodium alginate, dextrans, lectins, aminated gelatin, aminated pectin, hyaluronic acid, inulin, and combinations thereof.

9. The bioadhesive film of claim 1, wherein
said active agent is selected from anti-infective agents, spermicides, estrogens, progestins; deodorizers, and combinations thereof.

10. The bioadhesive film of claim 1, wherein said active agent is an anti-infective agents selected from antibiotics, sulfonamides, antivirals, antifungals, antiprotozoans, and mixtures thereof.

11. The bioadhesive film of claim 1, wherein said active agent is a spermicide selected from nonoxynol-9, octoxynol-9, benzalkonium chloride, ricinoleic acid, and phenol mercuric acetates, and combinations thereof.

12. The bioadhesive film of claim 1 wherein said active agent is one or more estrogen selected from conjugated estrogens, synthetic conjugated estrogens, esterified estrogens, 17b-estradiol, estradiol acetate, estropipate, and estradiol hemihidrate.

13. The bioadhesive film of claim 1 wherein said active agent is a progestin selected from medroxyprogesterone acetate, norethindrone, norgestel, megestrol acetate, progesterone, levonorgestrel, drospirenone, norgestimate, and methyltestosterone, and combinations thereof.

14. A bioadhesive film comprising:
65-90% polyvinyl alcohol by weight;
5-20% glycerin by weight;
5-8% Carbomer Homopolymer Type B USP/NF by weight; and
0.01-0.50% conjugated estrogen concentrate (16 mg/g) by weight.

15. The bioadhesive film of claim 14 comprising:
about 76.1 % Polyvinyl alcohol by weight;
about 16.25% glycerin by weight;
about 7.5% Carbomer Homopolymer Type B USP/NF by weight; and
about 0.15% conjugated estrogen concentrate (16 mg/g) by weight.

16. The bioadhesive film of claim 14, comprising:
about 72.35% polyvinyl alcohol by weight;
about 20% glycerin by weight;
about 7.5% Carbomer Homopolymer Type B USP/NF by weight; and
about 0.15% conjugated estrogen concentrate (16 mg/g) by weight.

17. The bioadhesive film of claim 14, further comprising an amount of pH buffer sufficient to adjust the film pH to about 7.4.

18. The bioadhesive film of claim 17, wherein said pH buffer is selected from tris(hydroxymethyl)-aminomethane and triethanolamine.

19. The bioadhesive film of claim 17 comprising:
about 68.81 % polyvinyl alcohol by weight;
about 14.69% glycerin by weight;
about 6.78% Carbomer Homopolymer Type B USP/NF by weight;
about 0.14% CE concentrate (16 mg/g) by weight; and
about 9.58% tris(hydroxymethyl)-aminomethane by weight.

20. The bioadhesive film of claim 17 comprising:
about 65.03% polyvinyl alcohol by weight;
about 17.98% glycerin by weight;
about 6.74% Carbomer Homopolymer Type B USP/NF by weight;
about 0.13% CE concentrate (16 mg/g) by weight; and
about 10.12% tris(hydroxymethyl)-aminomethane by weight.

21. The bioadhesive film of claim 17 comprising:
about 79.90% polyvinyl alcohol by weight;
about 9.42% glycerin by weight;
about 4.71% Carbomer Homopolymer Type B USP/NF by weight;
about 0.14% CE concentrate (16 mg/g) by weight; and
about 5.84% tris(hydroxymethyl)-aminomethane by weight.

22. The bioadhesive film of claim 17, comprising:
about 68.89% polyvinyl alcohol by weight;
about 14.71% glycerin by weight;
about 6.79% Carbomer Homopolymer Type B USP/NF by weight;
about 0.14% CE concentrate (16 mg/g) by weight; and
about 9.48% triethanolamine by weight.

23. The bioadhesive film of claim 17, comprising:
about 65.31% polyvinyl alcohol by weight;
about 18.05% glycerin by weight;
about 6.77% Carbomer Homopolymer Type B USP/NF by weight;
about 0.14% CE concentrate (16 mg/g) by weight; and
about 9.74% triethanolamine by weight.

24. The bioadhesive film of claim 17, comprising:
about 78.83% polyvinyl alcohol by weight;
about 9.29% glycerin by weight;
about 4.65% Carbomer Homopolymer Type B USP/NF by weight;
about 0.14% CE concentrate (16 mg/g) by weight; and
about 7.10% triethanolamine by weight.

25. A pharmaceutically acceptable bioadhesive film comprising:
About 65-95%, by weight film forming polymer selected from polyvinyl alcohol (PVA), polyethylene oxide, hydroxypropyl cellulose (HPC), hydroxyethyl cellulose (HEC), methylcellulose (MC), and hydroxypropyl methylcellulose (HPMC), copovidone, gelatin, maltodextrin, xanthan gum, guar gum, polymethacrylate, povidone, and mixtures thereof;
About 5-20%, by weight, plasticizer selected from glycerin, polyethylene glycols, propylene glycol, mannitol, sorbitol, dibutyl phthalate, tributyl citrate, dimethyl phthalate, pyrrolidones, and mixtures thereof and
Less than about 10%, by weight, bioadhesive agent selected from high-molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, and combinations thereof; and
a pharmaceutically active agent selected from anti-infective agents, spermicides, estrogens, progestins; deodorizers, and combinations thereof.
